(19) United States
(12) Patent Application Publication
(10) Pub. No.: US 2022/0323644 A1
(43) Pub. Date: Oct. 13, 2022

(54) METHOD OF TREATING OR REPAIRING AN ABDOMINAL WALL OPENING

(71) Applicant: The Board of Supervisors of Louisiana State University and Agricultural and Mechanical College, Baton Rouge, LA (US)

(72) Inventors: Ian HODGDON, New Orleans, LA (US); Michael COOK, New Orleans, LA (US); John PAIGE, New Orleans, LA (US); Jeffrey Carter, Metairie, LA (US)

(21) Appl. No.: 17/641,812

(22) PCT Filed: Sep. 9, 2020
(86) PCT No.: PCT/US2020/049952

§ 371 Date: Mar. 9, 2022

Related U.S. Application Data
(60) Provisional application No. 62/897,610, filed on Sep. 9, 2019.

(51) Int. Cl. A61L 27/36; A61F 2/00; A61L 27/38; A61L 27/54

(52) U.S. Cl. CPC A61L 27/362; A61F 2/0063; A61L 27/3687; A61L 27/3804; A61L 27/54; A61F 2240/001; A61L 2430/34; A61L 2300/412

(57) ABSTRACT

This invention is directed to systems and methods for treating a hernia by implanting a tissue graft material.

This application claims priority from U.S. Provisional Patent Application No. 62/897,610, filed on Sep. 9, 2019, which is incorporated by reference herein in its entirety.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to methods for treating a hernia with reduced complications by implanting a tissue graft material.

### BACKGROUND OF THE INVENTION

Hernia repair is the most common procedure performed with over one million surgeries in the United States each year. Synthetic mesh is used in over 80% of hernia repairs with 12% of patients developing a subsequent infection and complication involving the mesh. These complications have led to complex, severe injuries, dramatically impacting patients' lives and over $1 billion in settlements in the United States. In 2006 3.2 billion was spent on abdominal hernia repairs in the US alone.

### SUMMARY OF THE INVENTION

The present invention provides a method of preventing the development of a hernia within a subject at risk of developing a hernia, or a method of treating or repairing a hernia comprising: selecting a graft material; and implanting the graft material in contact with an opening in the abdominal wall, wherein the graft material promotes healing of the abdominal wall opening.

For example, aspects of the invention comprise a method of preventing the development of a hernia within a subject at risk of developing a hernia. In embodiments, the method comprises selecting a graft material, wherein the graft material comprises an epidermis and a dermis; removing the epidermis from the graft material through enzymatic processing, thereby providing a dermal graft material substantially comprising dermis; and implanting the dermal graft material in contact with an opening in the abdominal wall, wherein the dermal graft material repairs the abdominal wall opening, thereby preventing the development of a hernia in the subject.

Aspects of the invention are further directed towards a method of repairing a hernia. In embodiments, the method comprises selecting a graft material, wherein the graft material comprises an epidermis and a dermis; removing the epidermis from the graft material through enzymatic processing, thereby providing a dermal graft material; and implanting the dermal graft material in contact with an opening in the abdominal wall, wherein the dermal graft material repairs the abdominal wall opening.

In embodiments, the graft material comprises a full-thickness autologous skin graft.

Embodiments can further comprise a step of perforating the graft material to permit efflux of serous fluid.

Still further, embodiments can comprise the steps of collecting an epidermal cellular solution following enzymatic processing, wherein the epidermal solution substantially comprises the epidermis that was subjected to enzymatic processing; and applying the epidermal cellular solution to the abdominal wall opening, wherein the epidermal cellular solution promotes healing of the abdominal wall opening.

In embodiments, the enzymatic processing comprises exposure of the graft material to an enzymatic solution that is devoid of xenogeneic components, allogeneic components, or a combination thereof. For example, an enzyme used in the enzymatic processing is configured to disrupt disulfide bonds. Non-limiting examples of such enzymes comprise trypsin, dispase, collagenase, trypsin-EDTA, thermolysin, pronase, hyaluronidase, elastase, papain, pancreatin, DNAase, or a combination thereof. For example, the enzyme comprises a RECELL® Enzyme.

In embodiments, the dermal graft material comprises a cellular suspension of dermal cells capable of reproduction.

In embodiments, the step of implanting the dermal graft material comprises spraying the cellular suspension onto, in close proximity to, or adjacent to the abdominal wall opening. In embodiments, the step of implanting the dermal graft material comprises spraying the cellular suspension onto, in close proximity to, or adjacent to an orthopedic implant implanted into a subject.

In embodiments, the dermal graft material is aligned with the opening in the abdominal wall. Non-limiting examples of such opening comprises a surgical incision or debrided fascia.

In embodiments, dermal graft material promotes healing of abdominal fascial edges.

In embodiments, the dermal graft material is not actively affixed to the abdominal wall.

In embodiments, the dermal graft material is anchored to the abdominal wall by pressure, an adhesive, a clip, a tack, a suture, a staple, or a screw.

In embodiments, the dermal graft material is implanted over or ventral to the abdominal wall opening. In other embodiments, the dermal graft material is implanted under or dorsal to the abdominal wall opening.

Embodiments can further comprise substantially or completely closing the abdominal wall opening prior to implanting the dermal graft material. Other embodiments can comprise substantially or completely closing the abdominal wall opening after implanting the dermal graft material.

In embodiments, the abdominal wall opening is closed with synthetic mesh or biological mesh.

In embodiments, the abdominal wall opening comprises a surgical incision, such as an abdominal fascia incision. Thus, the abdominal wall opening can be caused by surgery. Non-limiting examples of such surgeries comprise laparotomy (celiotomy), stoma surgery, or repair of abdominal hernia.

In embodiments, the hernia comprises an incisional hernia, ventral hernia, umbilical hernia, epigastric hernia, lumbar hernia, inguinal hernia, diaphragmatic hernia, hiatal hernia, Spigelian hernia, or a parastomal hernia.

In embodiments, the subject at risk of developing a hernia comprises an obese subject, a subject afflicted with an infection, a subject who underwent a bowel resection, a subject who underwent colon surgery, a subject being administered corticosteroids, a subject who smokes, a subject who has chronic obstructive pulmonary disease or any combination thereof. For example, non-limiting examples of such infections comprise a surgical site infection, intra-abdominal infection, deep infections, or superficial abdominal infection.

In embodiments, the graft material comprises a mammalian tissue. The mammalian tissue can comprise endogenous growth factors. Non-limiting examples of such graft materials comprise a dehydrated tissue, decellularized tissue, cross-linked tissue, frozen tissue, cryopreserved tissue, fresh tissue, or any combination thereof.

In embodiments, the graft material can be implanted as a sheet, nanoparticle, powder, or injectable.

In embodiments, the graft material further comprises a synthetic mesh, biological mesh, or tissue scaffold. For example, the biological mesh comprises a mammalian tissue, non-limiting examples of which comprise a dermal matrix or a urinary bladder matrix. For example, the mammalian tissue scaffold comprises a collagen matrix.

Aspects of the invention are further directed towards a method of producing a dermal graft material. For example, embodiments comprise obtaining a graft material comprises an epidermis and a dermis; disaggregating epidermis from the graft material, wherein the step of disaggregating the epidermis from the graft material comprises subjecting the graft material to enzymatic processing, thereby providing a dermal graft material.

In embodiments, the graft material comprises full-thickness skin graft.

Still further, aspects of the invention are drawn towards a method of preventing cyst formation following implantation of a dermal graft material within a subject. For example, embodiments can comprise selecting a graft material, wherein the graft material comprises an epidermis and a dermis; removing the epidermis from the graft material through enzymatic processing, thereby providing a dermal graft material; and implanting the dermal graft material, wherein the enzymatic processing prevents cyst formation following implantation of the dermal graft material.

In embodiments, the dermal graft material comprises a cutis graft.

In embodiments, dermal graft material comprises an autologous dermal graft.

In embodiments, the graft material comprises a full-thickness skin graft.

Yet further, aspects of the invention are drawn to a method of treating an internal wound. In embodiments, for example, the method comprises selecting a graft material, wherein the graft material comprises an epidermis and a dermis; removing the epidermis from the graft material through enzymatic processing, thereby providing a dermal graft material; and implanting the dermal graft material over an internal wound of a subject.

In embodiments, the graft material comprises a cutis graft.

In embodiments, the graft material comprises an autologous dermal graft material.

For example, the internal wound comprises an injury to a tendon, a fistula, gastrotomy, enterotomy, colotomy, bile duct injury, bladder injury, ureteral injury, arterial or venous injuries, serosal tears, or a combination thereof. For example, the fistula comprises an anal fistula, colorectal fistula, enterocutaneous fistula, choledochocutaneous fistula, cholecystocutaneous fistula, gastrocutaneous fistula, esophagocutaneous fistula, gastroatmospheric fistula, enteroatmospheric fistula, coloatmospheric fistula, rectoatmospheric fistula, gastrogastric fistula, gastroenteric fistula, gastrocolic fistula, enteroenteric fistula, enterocolic fistula, colovaginal fistula, colovesical fistula, rectovaginal fistula, rectovesical fistula, colourethral fistula, rectourethral fistula, tracheoesophageal fistula, bronchoesophageal fistula, tracheopleural fistula, bronchopleural fistula, aortoenteric fistula, or a combination thereof.

Still further, aspects of the invention are drawn to a method of promoting healing of an opening in the abdominal wall. For example, embodiments comprise obtaining a graft material, wherein the graft material comprises an epidermis and a dermis; removing the epidermis from the graft material through enzymatic processing, thereby providing a dermal graft material; and implanting the dermal graft material in a subject, wherein the dermal graft material is implanted in contact with the opening in the abdominal wall, and wherein the dermal graft material promotes healing of the abdominal wall opening.

Aspects of the invention are also drawn towards a method of promoting fascial healing. For example, embodiments comprise obtaining a graft material, wherein the graft material comprises an epidermis and a dermis; removing the epidermis from the graft material through enzymatic processing, thereby providing a dermal graft material; and implanting dermal graft material in a subject, wherein the dermal graft material is implanted approximately to a defective region in the fascia, and wherein the dermal graft material promotes healing of the defect in the fascia.

Aspects of the invention are also drawn towards a method of preventing hernia recurrence. For example, embodiments comprise obtaining a graft material, wherein the graft material comprises an epidermis and a dermis; removing the epidermis from the graft material through enzymatic processing, thereby providing a dermal graft material; and implanting the dermal graft material in a subject, wherein the dermal graft material is implanted in contact with an opening in the abdominal wall, and wherein the dermal graft material prevents hernia recurrence.

Aspects of the invention are also drawn towards a kit for producing a dermal graft. In embodiments, the kit comprises, the kit comprises an enzymatic solution configured to disaggregate the epidermis from a full-thickness skin graft, thereby producing a dermal graft material; and instructions for use.

Further, aspects of the invention are directed towards a method of treating a medical condition with a regenerative cell suspension. For example, in embodiments, the method comprises obtaining an epidermal solution, wherein the epidermal solution comprises autologous epidermal cells from a subject suffering from the medical condition; and applying the epidermal solution to an area afflicted with the medical condition, wherein the epidermal solution treats the medical condition.

Further, embodiments can comprise implanting a dermal graft material to the area afflicted with the medial condition, wherein the dermal graft material and the epidermal solution treat the medical condition.

Aspects of the invention are also drawn towards a method of creating a regenerative cell suspension for the treatment of a medical condition. For example, the method comprises applying an enzymatic solution to an epidermis of a subject suffering from the medical condition, wherein the enzymatic solution is configured to disaggregate the epidermis from a dermis of the subject; permitting the enzymatic solution to disaggregate the epidermis from the dermis to form an epidermal solution; and collecting the epidermal solution, wherein the collected epidermal solution forms the regenerative cell suspension

In embodiments, the enzymatic solution is applied to epidermis from an autologous, full-thickness skin graft obtained from the subject.

In embodiments, the medical condition comprises chronic wounds, exfoliative skin diseases, a surgical wound, or a combination thereof.

Still further, aspects of the invention are drawn towards a regenerative cell suspension configured to treat a medical condition. For example, the regenerative cell suspension can comprise an epidermal solution, wherein the epidermal solution comprises autologous epidermal cells from a subject afflicted with the medical condition. In embodiments, the medical condition comprises chronic wounds, exfoliative skin diseases, a surgical wound, or a combination thereof.

Also, aspects of the invention are drawn towards a method of preventing the development of a hernia within a subject at risk of developing a hernia. In embodiments, the method comprises selecting a graft material, wherein the graft material comprises an epidermis and a dermis; removing the epidermis from the graft material through enzymatic processing, collecting an epidermal solution following enzymatic processing, wherein the epidermal solution comprises the epidermis that was subjected to enzymatic processing; and applying the epidermal solution to an abdominal wall opening, wherein the epidermal solution promotes healing of the abdominal wall opening.

Another aspect includes an enzymatic debridement system for use in creating a dermal graft from a graft material when the graft material comprises an epidermis and a dermis. In various embodiments, the enzymatic debridement system comprises an enzymatic solution configured to remove substantially all of the epidermis from the graft material when exposed thereto. In embodiments, the enzymatic solution is devoid of xenogenic components, allogentic components, or a combination thereof. In certain embodiments, an enzyme used in the enzymatic processing is configured to disrupt disulfide bonds. The enzyme can comprise trypsin, dispase, collagenase, trypsin-EDTA, thermolysin, pronase, hyaluronidase, elastase, papain, pancreatin, DNAase, or a combination thereof. In certain embodiments, the enzyme comprises a RECELL® Enzyme.

The graft material can comprise a mammalian tissue. In embodiments, the mammalian tissue comprises endogenous growth factors. The graft material can comprise a dehydrated tissue, decellularized tissue, cross-linked tissue, frozen tissue, cryopreserved tissue, fresh tissue, or any combination thereof. In certain embodiments, the dermal graft material comprises a cutis graft. In embodiments, the graft material comprises an autologous graft. The graft material can comprise a full-thickness skin graft. In one embodiment, the enzymatic debridement system comprises a lyophilized powder configured to extend a shelf life of the resultant dermal graft.

In any of the various exemplary embodiments disclosed herein, the dermal graft comprises a tensile strength that is substantially equivalent the tensile strength of a full thickness skin graft. The dermal graft can comprise a tensile strength of between about 50 N/cm to about 800 N/cm. In embodiments, the dermal graft comprises a tensile strength of up to about 700 N/cm. The dermal graft can comprise a tensile strength of about 100 N/cm, about 200 N/cm, about 300 N/cm, about 400 N/cm, about 500 N/cm, about 600 N/cm, about 700 N/cm, about 800 N/cm, about 900 N/cm, or about 1000 N/cm. In one embodiment, the dermal graft comprises a tensile strength of about 525 N/cm, about 550 N/cm, about 575 N/cm, about 600 N/cm, about 625 N/cm, about 650 N/cm, about 675 N/cm, or about 700 N/cm.

Other objects and advantages of this invention will become readily apparent from the ensuing description.

### DETAILED DESCRIPTION OF THE INVENTION

Two million laparotomies are performed annually in the United States with incisional hernias (IH) being a major complication. A laparotomy can refer to a surgical incision into the abdominal cavity, such as for diagnosis of a disease or condition or in preparation for surgery. The incidence of IH following laparotomy averages 12% (Franz, 2008a, Bosanquet et. al, 2015) but reaches 73% in high risk populations (Veljkovic et. al, 2010). This leads to 400,000 new IH per year. These hernias pose a major burden on the healthcare system, patients, and surgeons. IH repair (IHR) costs $6-10 billion per year (Hernia Repair Devices and Consumables). Patients undergoing IHR risk complications including surgical site infection (20-30%) and hernia recurrence (20-30%) (Goodenough et. al, 2015). Surgeons performing IHR frequently use synthetic mesh, which exposes them to mesh-related malpractice suits (Muysoms et. al, 2015) (ConsumerSafety.org, 2018).

Prior to our work, no clinically-proven strategies have been identified to reduce IH formation rates. At least one study has shown that changing the technique of closure can decrease formation, but no methods of using a biologic or chemical means have been shown.

As an alternative to synthetic mesh, which is often used in hernia repairs, autologous skin can be taken from the patient and used as a graft during the hernia repair. Referring to the Examples, data indicates that patients treated with such autologous skin graft material show a reduction in early complications over 11 months. Without being bound by theory, long term complications such as recurrence are reduced following treatment with the autologous skin graft material, and the long term outcomes of treatment with the autologous skin graft material are improved or at least comparable to those of synthetic mesh.

As a part of the procedure for implanting autologous skin graft material, the epidermis can be removed from the dermis to prevent seroma or epidermal cyst formation. In embodiments, the epidermal/dermal dissociation is performed mechanically. In certain embodiments, this step can be performed with an electrocautery scratch pad, a blade, or a combination thereof. However, such mechanical separation can lead to lead to graft damage and can be time consuming. Mechanical separation can require about 20 minutes of dedicated surgeon time for a graft that is about 100 cm² in size.

In alternate embodiments, the dermis and epidermis are dissociated chemically, such as through enzymatic processing. Without being bound by theory, such embodiments will reduce operating room time, reduce long-term complications, improve surgeon acceptance of the technic, or a combination thereof. For example, embodiments herein provide a time efficient method for supplying a cellular cover to a tissue in a clinical setting. That is, cells are available when needed at the time of surgery. This is achievable because there is a very short preparation period of the cells, thus allowing grafting to be performed pen-operatively or in the rooms of a specialist physician or General Practitioner.

The invention provides methods to promote fascial healing, prevent hernia development, repair a hernia, or prevent hernia recurrence. In embodiments, the methods comprise selecting a graft material, such as one comprising an epidermis and a dermis or derivative thereof, removing the epidermis from the graft material to create a dermal graft, and implanting the dermal graft material in contact with, in close proximity to, or adjacent to an opening in the fascia or abdominal wall. In embodiments, the epidermis is removed from the dermal layer through enzymatic processing.

The invention provides methods and embodiments to eliminate the burdens associated with mesh implants, for example, hernia recurrence, infection, and erosion. Such burdens can be costly to repair.

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

### Abbreviations and Definitions

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Hernia

Aspects of the invention are directed towards methods of healing an opening in the fascia, such as an incision or a hernia. The term “fascia” refers to a sheet of fibrous tissue that envelops the body beneath the skin. It also encloses muscles or groups of muscles. In embodiments, an opening or incision in the fascia can be prevented or treated as described herein. For example, the opening can comprise incised fascia, that is tissue of the fascia that has been cut, such as by an incision or electrocautery, and excised fascia, that is tissue of the fascia that has been removed by any means.

Aspects of the invention are also directed towards methods of healing an opening in the abdominal wall, such as an abdominal incision or an abdominal hernia. The abdominal wall represents the boundaries of the abdominal cavity, and is split into the posterior (back), lateral (sides), anterior (front) walls, superior (top or towards the head), and inferior (bottom or towards the pelvis). In human anatomy, the layers of the abdominal wall are (from superficial to deep) skin, subcutaneous tissue; fascia (Camper's fascia and Scarpa's fascia); muscle (external oblique abdominal muscle; internal oblique abdominal muscle; rectus abdominis; transverse abdominal muscle; pyramidalis muscle; transversalis fascia; extraperitoneal fat; and peritoneum. It also includes the linea alba, which runs from the xiphoid process to the pubic symphysis in the midline of the abdomen and is a coalescence of several layers of the abdominal wall. The superior boundary of the abdominal cavity is comprised of the diaphragm. The inferior boundary of the abdominal cavity is comprised of the pelvic floor.

In embodiments, the opening, such as caused by a surgical incision, can result in a hernia. Hernias can also be congenital, or can occur without being the result of surgery. These include inguinal hernias, sports hernias, and Spigelian hernias, for example.

The term “hernia” can refer to a protrusion of a part or structure through the tissues normally containing it. Hernias are typically named for the area where the protrusion occurs. For example, umbilical hernia is a protrusion of fat or viscera through the abdominal wall at the level of the umbilicus. As another example, an “abdominal hernia” can refer to a protrusion through or into any part of the abdominal wall, such as the case when the intestines extrude through a weakened area in the abdominal wall. The abdominal wall boundaries are the diaphragm superior, pelvis inferior, spine posterior, abdominal wall muscular lateral and anterior. For example, an abdominal wall hernia can refer to a protrusion of abdominal organ or fat protrusion through the boundary.

The most common types of hernias are inguinal (groin), incisional (resulting from an incision), femoral (groin), umbilical (belly button), parastomal, and hiatal/diaphragmatic (upper abdomen). Non-limiting examples of other types of hernias comprise lumbar, diaphragmatic, ventral, postoperative, epigastric, Spigelian, weakness in the pelvic floor (such as obturator hernia), or generally any abdominal wall related hernia. Non-limiting examples of non-incisional hernias comprise inguinal hernias, spigelian hernias, sports hernias, lumbar hernia, femoral hernia, diaphragmatic hernia, hiatal hernia, and/or obturator hernia. The skilled artisan will recognize that in the most general interpretation, a hernia can occur in any hollow body organs and/or said natural and/or said artificial orifices and/or said spaces and/or said post-operative spaces.

For example, embodiments of the invention are particularly suited for preventing or treating a parastomal hernia. A parastomal hernia is a type of incisional hernia that allows protrusion of abdominal contents through the abdominal wall defect created during ostomy formation. Unlike a hernia development in a surgical incision for which the fundamental problem is healing between tissues that have been approximated, ostomy creation introduces an abdominal wall defect for which no healing is expected. A parastomal hernia forms as the defect is continually stretched by the forces tangential to its circumference. The reported incidence of parastomal hernia varies widely and is related to the type of ostomy constructed, the duration of follow-up after ostomy construction, and the definition used to identify parastomal hernia. The incidence of parastomal hernia is reported as ranging from 0 to 50 percent, depending upon the type of ostomy.

Embodiments of the invention are particularly suited for preventing or treating an incisional hernia. An incisional hernia refers to a protrusion of tissue that forms at the site of a surgical incision or healing surgical scar. The term “surgical incision site” can refer to the body or tissue surface to which a surgical incision is to be made or has been made, as well as the immediate area adjacent to or in close proximity to the incision. The “surgical incision site” can be referred to as an “opening” in a tissue. This immediate area extends in all directions beyond the incision. For example, the immediate area can extend by about 2 to 12 inches beyond the incision. For example, an abdominal hernia can result from an incision causing an opening in the abdominal wall, and thus can be referred to as an incisional hernia. Surgeries that can result in incisional hernias, for example, comprise laparotomy (celiotomy), laparoscopy, stoma surgery, or repair of abdominal hernia. A hernia can also form from devascularization or weakening of the abdominal wall, such as from the surgery itself or a surgical site infection secondary to the surgery.

Embodiments of the invention can also be suited for treating the hernia and the area in proximity to the hernia. For example, the area in proximity to hernia can comprise the abdominal wall musculature and fascia, diaphragm, and/or pelvis.

Although hernias can occur in any subject, particular groups of individuals are especially susceptible to hernia development. For example, subjects at risk of developing a hernia comprise those who are obese and/or afflicted with diabetes, afflicted with an infection (such as a surgical site infection, intra-abdominal infection, deep tissue infection, or superficial abdominal infection), malnourished, undergoing preoperative chemotherapy, of advanced age, pregnant, afflicted with connective tissue disease, suffering from chronic cough, underwent intraoperative blood transfusion, underwent a surgery (such as a bowel resection, colon surgery, or emergency surgery), are being administered corticosteroids, smokes, has chronic obstructive pulmonary disease, has emphysema, has peripheral vascular disease, has diabetes mellitus Type 1 or Type 2, or any combination thereof. Other risk factors include, but are not limited to, congestive heart failure, renal failure, liver failure (especially with ascites), patients with constipation or BPH (intentional increase in abdominal pressure to void or defecate), patients with upper or lower extremity amputations or weakness (via increased dependence on core musculature). Patients with jobs requiring heavy lifting, or caring for invalid family members can also predispose to hernia formation. According to Mayo clinic, being male and being Caucasian increase your risk for hernia, as well as family history of hernias.

Embodiments of the invention are useful to treat complicated hernias. Complicated hernias, for example, include those that are large, involve previously placed mesh, are infected, or are performed during a contaminated or clean contaminated procedure. Complicate hernias also include bowel ischemia (incarceration and/or strangulation) and recurrent hernias, for example.

Embodiments of the invention can be used to promote healing of or treat an internal wound, such as a wound within the body of a subject. Embodiments herein allow for faster healing of internal wounds, whether caused naturally, by surgery, or trauma, for example, thereby reducing trauma for patients during the phase of their medical care. For example, the internal wound can be an injury to a tendon, a fistula, gastrotomy, enterotomy, colotomy, bile duct injury, bladder injury, ureteral injury, arterial or venous injuries, serosal tears, or a combination thereof. Non-limiting examples of the fistula comprises an anal fistula, colorectal fistula, enterocutaneous fistula, choledochocutaneous fistula, cholecystocutaneous fistula, gastrocutaneous fistula, esophagocutaneous fistula, gastroatmospheric fistula, enteroatmospheric fistula, coloatmospheric fistula, rectoatmospheric fistula, gastrogastric fistula, gastroenteric fistula, gastrocolic fistula, enteroenteric fistula, enterocolic fistula, colovaginal fistula, colovesical fistula, rectovaginal fistula, rectovesical fistula, colourethral fistula, rectourethral fistula, tracheoesophageal fistula, bronchoesophageal fistula, tracheopleural fistula, bronchopleural fistula, aortoenteric fistula, or a combination thereof

Graft Material

Aspects of the invention are directed towards methods of implanting a graft material, such as a dermal graft material in a subject to promote fascial healing, repair a hernia, prevent hernia development, prevent hernia recurrence, or prevent the failure of hernia repair.

The term “graft material” can refer to a material that can be placed on, attached to or inserted into a bodily part. The material can be a tissue graft material which comprises tissue and/or processed tissue. The graft material can comprise a tissue sample. For example, the graft material can be a full thickness skin graft. As another example, the graft material can be a dermal graft material, which substantially comprises the dermis. The tissue graft material can further comprise additional compositions, such as synthetics or biological compositions, as described herein. The graft material can be a mammalian tissue or derivative thereof. In embodiments, the mammalian tissue comprises an epidermal layer and a dermal layer or derivatives thereof. A tissue derivative is prepared from a tissue, such as a mammalian tissue, through physical and/or chemical treating of the natural tissue to produce a derivative tissue that retains the natural structure and/or basic characteristics of the natural tissue. For example, the tissue can be dehydrated, such as chemically dehydrated or freeze-dried; decellularized; cross-linked; frozen; cryopreserved; fresh; decontaminated; cleaned; or any combination thereof, thereby producing a tissue derivative. A tissue derivative may also be prepared from cells isolated from the tissue. For example, as described herein, an epidermal solution can comprise a solution comprising epidermal cells isolated from a full thickness skin graft.

According to this method, tissue (preferably of an autologous nature) is harvested from a patient by means known in the art of tissue grafting. Preferably this is achieved by taking a tissue biopsy. With the harvesting of the biopsy consideration must be given to the depth of the biopsy and the surface area size. The depth and size of the biopsy influence the ease at which the procedure can be undertaken and speed with which a patient recovers from the procedure. In an embodiment, the donor site can be chosen to appropriately match the recipient site, for example post-auricular for head and neck, thigh for lower limbs, inner-upper-arm for upper limbs, or palm for sole or vice-versa.

Once a biopsy has been harvested from a patient, the tissue sample is subjected to physical and/or chemical dissociating means capable of dissociating cellular stratum in the tissue sample. For example, a graft material comprising an epidermis and a dermis is selected, and the epidermis and dermis are subsequently separated. For example, the dissociating means can be either a physical or a chemical disruption means. Physical dissociation means can include, for example, scraping the tissue sample with a scalpel, mincing the tissue, physically cutting the layers apart, or perfusing the tissue. Chemical dissociation means might include, for example, digestion with enzymes. Exemplary enzymes include trypsin, dispase, collagenase, trypsin-EDTA, thermolysin, pronase, hyaluronidase, elastase, papain, pancreatin, DNAase, or a combination thereof. Non-enzymatic solutions for the dissociation of tissue can also be used. In an embodiment where the epidermis and dermis are dissociated from a full thickness skin graft by enzymatic means, the resulting compositions can comprise a dermal graft material, substantially comprising dermis, and an epidermal solution, substantially comprising epidermal cells.

In embodiments, the graft material comprises an allograft. The term “allograft” can refer to a tissue graft from a donor of the same species as the recipient but not genetically identical. For example, the allograft can comprise a cutis allograft or skin allograft, such as a full-thickness skin allograft. In other embodiments, the graft material comprises an autograft. For example, the autograft can comprise a cutis autograft or skin autograft, such as a full-thickness skin autograft. The term “autograft” can refer to a graft of tissue from one point to another on the same individual's body.

In embodiments, the graft material can be a cutis graft or a skin graft. The term “skin graft” can refer to one or more of the individual components of the skin. In embodiments, the skin graft does not comprise the entire skin. Such components are well known in the art and include an epidermis, a dermis, or any combination thereof. A skin graft can include a split-thickness graft or a full-thickness graft. A “split-thickness graft” or “full thickness skin graft” can be a skin graft that comprises the epidermis or alternatively the dermis. For example, a split-thickness graft can comprise the dermis, but not the epidermis. For example, the epidermis can be removed either mechanically, chemically, or enzymatically, and the dermis (i.e., dermal graft) is then used to repair the hernia or internal wound. In certain embodiments, the epidermal cells can be applied to the hernia or internal wound to promote healing. In certain embodiments, a split-thickness graft additionally comprises a portion of the dermis or just the dermis. A “full-thickness graft” or “full-thickness skin graft” can be a skin graft that comprises the epidermis and dermis. In certain embodiments, a full-thickness skin graft additional includes at least a portion of the hypodermis.

The terms “dermal tissue graft,” “dermal graft,” “dermis graft,” and “dermal skin graft” and other comparable words/phrases can be used interchangeably to refer to a graft material substantially comprised of the dermis. In certain embodiments, a dermal graft includes a skin graft in which the epidermis has been removed. By way of non-limiting examples, the epidermis can be removed or dissociated through physical, chemical, or enzymatic means. The epidermis can be removed mechanically, chemically, enzymatically, or a combination thereof. In certain embodiments, epidermis is removed from a skin graft enzymatically to permit the formation of a dermal graft material.

Non-limiting examples of physical dissociation include, scraping the tissue sample with a scalpel, mincing the tissue, physically cutting the layers apart, perfusing the tissue, or a combination thereof. Non-limiting examples of chemical dissociation include, for example, digestion with enzymes (i.e., enzymatic means). Exemplary enzymes appropriate for such digestion include, but are not limited to trypsin, dispase, collagenase, trypsin-EDTA, thermolysin, pronase, hyaluronidase, elastase, papain, pancreatin DNAase, or a combination thereof. In alternative embodiments, non-enzymatic solutions are used for the dissociation of tissue.

Enzymatic processing can include subjecting the graft material to a cell dissociation treatment. Exemplary cell dissociation treatments are provided in U.S. Pat. Nos. 9,029,140 and 9,078,741, which are incorporated herein by reference in their entirety. Briefly, dissociation of a graft material or tissue sample, such as a full thickness skin graft, can be achieved by placing the graft material in a pre-warmed enzyme solution containing an amount of enzyme sufficient to dissociate cellular stratum in the graft material. In an embodiment, a graft material is provided, such as a dermal graft material, and a cellular suspension is provided, such as a epidermal cellular suspension. Tissue/cellular dissociation can be achieved, for example, by using a trypsin solution, however, any other enzymes that cause cells to become detached from other cells or from solid surfaces can be used for this purpose. Non-limiting examples of such enzymes comprise dispase, collagenase, DNAase, trypsin-EDTA, thermolysin, pronase, hyaluronidase, pancreatin, elastase and papain. In certain embodiments, the enzyme solution comprises an enzymatic solution used in the RECELL® System (available from Avita Medical, Valencia, Calif.). When the enzyme used is trypsin the enzyme solution used in the method can be calcium and magnesium free. In one embodiment, the solution comprises calcium and magnesium ion free phosphate buffered saline.

In embodiments, the tissue can be processed before or during a surgical procedure. For example, the tissue can be processed, such as enzymatically processed or mechanically processed, before a surgical procedure or during a surgical procedure. For example, the tissue can be placed into a processing solution for a period of time while a surgical procedure is underway. In such embodiments, the tissued can be placed into a processing solution by a medical profession within the surgical suite itself.

In embodiments where the tissue biopsy is derived from a patient's skin (such as a skin graft comprising epithelial-dermal cells) the amount of trypsin used in the method can be between about 5 and 0.1% trypsin per volume of solution. In embodiments, the trypsin concentration of the solution is about 2.5 to 0.25%. The concentration of trypsin can be about 0.5%.

The time period over which the tissue sample or graft material is subjected to the trypsin solution can vary depending on the size of the biopsy sample taken. The tissue sample or graft material can be placed in the presence of the trypsin solution for sufficient time to weaken the cohesive bonding between the tissue stratum. For example, where the tissue sample is taken from a patient's skin the sample can be placed in trypsin for a 5 to 60 minute-period. The tissue sample can be immersed in the trypsin solution for between 10 and 30 minutes. In certain embodiments, the tissue sample or graft material is immersed in the trypsin solution for about 15 to about 20 minutes.

After the tissue sample or graft material has been immersed in the trypsin solution for an appropriate amount of time, the graft material can be removed from the trypsin and washed with nutrient solution. Washing the tissue sample or graft material can involve either partial or complete immersion of the treated sample or graft material in the nutrient solution. Alternatively, the wash solution can be dripped on the tissue sample or graft material in sufficient volume to remove and/or significantly dilute any excess trypsin solution from the surface of the sample or graft material. In embodiments, dilution leads to less than 0.05% trypsin in the nutrient solution.

The nutrient solution used in the method can be capable of significantly reducing or removing the effect of the trypsin. In embodiments, this is achieved by dilution, neutralization, or a combination thereof. The nutrient solution used in the method can also comprise any one or more of the following characteristics (i) free of at least Xenogenic serum and/or xenogenic components, (ii) free of allogenic components, (iii) capable of maintaining the viability of the cells until applied to a patient, and (iv) suitable for direct application to a region on a patient undergoing tissue grafting. For example, according to the method of preparation and treatment the cells used in a graft are suspended in a nutrient solution free of xenogenic serum. That suspension is then placed directly onto the recipient site. The solution can comprise a basic salt solution, or the solution can comprise a more complex nutrient solution. In embodiments, the nutrient solution comprises a physiological saline. In embodiments, a “physiological saline” can be a solution that is free of all serum while containing various salts that resemble any one or more of the substances found in body fluids. Phosphate or other non-toxic substances can also buffer the solution in order to maintain the pH at approximately physiological levels. A suitable nutrient solution that is can comprise Hartmann's solution.

In embodiments, after application of the nutrient solution to the tissue sample, the cellular stratum of the sample can be separated. This separation can permit cells capable of reproduction to be removed from the cellular material and suspended in the nutrient solution. Where the tissue sample or graft material comprises skin, the dermis and epidermis can be separated to allow access to the dermal-epithelial junction of both surfaces.

Cells capable of reproduction can then be removed from the separated stratum by any means known in the art. In certain embodiments, the reproductive cells are scraped off the surface of the stratum. An exemplary instrument for performing such scraping can be a scalpel. Cells capable of reproduction within the dermal-epithelial junction include but are not limited to keratinocyte basal cells, Langerhans cells, fibroblasts, and melanocytes. Following release of the cells from the tissue sample the cells capable of reproduction can be suspended in the nutrient solution. In embodiments, only a small volume of nutrient solution is applied to the tissue sample during this harvesting step in an effort to prevent the suspension from becoming overly fluid therein, which can provide difficulties in applying the suspension to the graft.

To avoid excessively large cellular congregates in the cellular suspension the suspension can be filtered. Any filter capable of separating excessively large cellular congregates from the suspension may be used. In embodiments, the filter size is between about 50 μm and about 200 μm. The filter size can be between about 75 μm and about 150 μm. In one embodiment, the filter size is about 100 μm.

Prior to application to the graft site or immediately after filtering, the cellular suspension can be diluted to produce an appropriate cell density suitable for the purpose to which the suspension is to be used.

According to an aspect of the invention there is provided a cell suspension produced by the methods described herein. In embodiments, the cell suspension is an aqueous cell suspension. For example, the aqueous cell suspension includes one or more viable cells. The skilled artisan will recognize that any of a variety of cell types can be included in the cell suspension. For example, the cell suspension can comprise stem cells, epidermal cells, melanocytes, merkel cells, Langerhans cells, growth factors, and the like. The aqueous cell suspension can comprise a homogenous population of cells, or can comprise a heterogenous population of cells. The cell suspension provided by this method is highly suitable for tissue regeneration and grafting techniques. An important advantage of utilizing such a suspension in grafting technology is that it can be used to greatly expand the area or volume of a wound that can be treated quickly by in situ multiplication of a limited number of cells. The number and concentration of cells seeded onto graft site may be varied by modifying the concentration of cells in suspension, or by modifying the quantity of suspension that is distributed onto a given area or volume of the graft site.

Without wishing to be bound by theory, by suspending cells in a nutrient solution which is at least (i) free of xenogenic serum and components, (ii) capable of maintaining the viability of the cells until applied to a patient and (iii) is suitable for direct application to a region on a patient undergoing tissue grafting, the outcome of patient grafts is improved. Without wishing to be bound by theory, an explanation for this appears to be attributable to the removal of xenogenic serum and more preferably all serum from the cell suspension. Xenogenic serum is a common additive in grafting culture medium and is well known to cause potential infective and hypersensitivity problems. Such serum is however generally required for the in vitro expansion of the cells and to neutralize the action of the enzyme if the enzyme used is trypsin. In embodiments, the nutrient solution used in the present invention does not require such serum because the cell population within the suspension is not expanded prior to application to the graft site. Rather cellular multiplication is encouraged in the patient rather than in an in vitro system. When trypsin is used neutralization is achieved by other means.

Another feature of the cellular suspension produced as described herein is that the graft cells are more viable as they are harvested in a nutrient solution as distinct from prior art cell harvesting procedures which utilize techniques where the cells are harvested whilst exposed to powerful digestive enzymes for excessive periods of time. When the cells are exposed to such enzymes for excessive periods of time the viability of the cellular suspension decreases.

In various embodiments, the cell suspension is highly suitable for tissue regeneration and grafting techniques. One advantage of utilizing such a suspension in grafting technology is that it can be used to greatly expand the area or volume of a wound that can be treated quickly by in situ multiplication of a limited number of cells. The number and concentration of cells seeded onto graft site may be varied by modifying the concentration of cells in suspension, or by modifying the quantity of suspension that is distributed onto a given area or volume of the graft site.

In embodiments, tissue grafts can be applied to the subject in various forms. As discussed above, graft material can be applied as a cellular suspension. For example, a cellular suspension substantially comprise epidermal cells can be applied. In another example, a cellular suspension substantially comprising dermal cells can be applied. In another example, a cellular suspension substantially comprising dermal cells and epidermal cells can be applied. In alternate embodiments, the tissue graft can be applied in membrane form, for example as a sheet or sheet-like. Further, the tissue graft can be applied in micronized form or powdered form, such as produced when membrane tissue has been cryomilled and sieved for particulate sizing. For example, micronized form grafts can be produced using 180 and 25 μm sieves for particulate sizing. The micronized or powdered graft material can be spread or scattered over an opening, such as an abdominal fascia incision that has been closed with suture. Alternatively, the micronized tissue can be reconstituted in a solution, such as a saline solution, and administered to patients as a flowable or injectable material. For example, the micronized graft material can be solubilized/dissolved and injected within 4-cm of an abdominal fascia incision that has been closed with suture.

As discussed herein, the graft material can be provided as a flat sheet or sheet-like form, such as in membrane form, or as a nanoparticle or powder, which can be referred to as micronized form. The terms “sheet” and “sheet-like,” as used herein, generally refer to a broad, relatively thin, surface or layer of a material. Such sheets can, but may not, be relatively flexible, and may be flat or uniform in thickness or may vary in thickness across their surface. The micronized form can be resuspended in a solution, such as water or saline solution, prior to implantation or administration to the subject. The resuspended micronized form or powder can be referred to as a slush or slurry.

In embodiments, the graft material can further comprise compositions which provide enhanced mechanical properties, functionality, and/or structure to the graft material. For example, the compositions can provide additional support to the graft material and/or the weakened or damaged tissue, or can function as a scaffold for endogenous cells to populate so to promote healing. As another example, the composition can allow the graft to be more durable than a graft that does not contain the composition and thus prevent graft failure due to mechanical forces. Such compositions can comprise a synthetic mesh, a biological mesh, or a tissue scaffold. For example, the mesh can aid in incorporation of the graft, and can prevent infection of a graft. In an embodiment, the epidermal cellular solution can be applied to a mesh.

Synthetic mesh are man-made compositions formed by the polymerization of a variety of monomers, such as macromolecules comprising polyacrylic acid, polyaspartic acid, polytartaric acid, polyglutamic acid, polyfumaric acid, and so on as well as their salt forms (such as sodium salt and potassium salt). Non-limiting examples of synthetic polymers comprise cyanoacrylate. The synthetic mesh can comprise polyglactin (such as Vicryl mesh), e-PTFE, polyprolylene, polyester, polyglycolic, polyester/collagen, polypropylene/PG910, polypropylene/e-PTFE, polypropylene/cellulose, polypropylene/PVDF, polypropylene/sodium hyaluronate, polypropylene/polyglecaprone, polypropylene/titanium, polypropylene/omega 3, BioA, and the like. See, for example, Gillern, Suzanne, and Joshua I S Bleier. “Parastomal hernia repair and reinforcement: the role of biologic and synthetic materials.” Clinics in colon and rectal surgery 27.04 (2014): 162-171, which is incorporated by reference herein in its entirety.

Mesh made of synthetic materials can be found in knitted mesh or non-knitted sheet forms. The synthetic materials used can be absorbable, non-absorbable or a combination of absorbable and non-absorbable materials.

Biological mesh are made of non-placental mammalian tissue, such as skin, bladder, or intestine, that has been processed, cross-linked, chemically treated, and/or disinfected to be suitable for use as an implanted device. The biological mesh may or may not be absorbable. The majority of tissue used to produce these mesh implants are from human, pig (porcine) or cow (bovine) source. Biological mesh can comprise biological compositions such as acellular dermal matrix or urinary bladder matrix. See, for example, Gillern, Suzanne, and Joshua I S Bleier. “Parastomal hernia repair and reinforcement: the role of biologic and synthetic materials.” Clinics in colon and rectal surgery 27.04 (2014): 162-171, which is incorporated by reference herein in its entirety. For example, the biological mesh can comprise a mesh that was living tissue of human or animal origin, rendered acellular, and comprised of either cross-linked on non-crosslinked proteins. The biological mesh can be partially or completely resorbed.

Biological mesh can also be formed by the polymerization of natural polymers. Natural polymers occur in nature and can be extracted, such as polysaccharides or proteins. Non-limiting examples of polysaccharides comprise chondroitin sulfate, heparin, heparan, alginic acid (i.e., alginate), hyaluronic acid, dermatan, dermatan sulfate, pectin, carboxymethyl cellulose, chitosan, melanin (and its derivatives, such as eumelanin, pheomelanin, and neuromelanin), agar, agarose, gellan, gum, and the like as well as their salt forms (such as sodium salt and potassium salt). Non-limiting examples of proteins comprise collagen, alkaline gelatin, acidic gelatin, gene recombination gelatin, and so on.

A tissue scaffold refers to a composition which can act as a structural scaffold, such as a scaffold by which viable cells can readily populate. The term “viable cell” can refer to a cell that is alive and capable of growth, proliferation, migration, and/or differentiation. For example, an epidermal cellular solution can comprise viable epidermal cells. For example, a tissue scaffold can comprise matrices, such as collagen matrix. The graft material can be mixed with the matrices and the resulting admixture can be applied on top of (overlay) an opening that has been closed with a suture. In some embodiments, cells from the native tissue (e.g., the host subject) can migrate into the tissue scaffold and readily repopulate the graft (and thus promote healing). In embodiments, the graft can be seeded with viable cells so as to repopulate the graft with the viable cells prior to implantation.

In embodiments, the graft is non-absorbable or substantially non-absorbable, which will remain in the body indefinitely and is considered a permanent implant. It is used to provide permanent reinforcement to the repaired hernia.

In other embodiments, the graft is absorbable or substantially absorbable, which will degrade over time. It is not intended to provide long-term reinforcement to the repair site. As the material degrades, new tissue growth is intended to provide strength to the repair. In embodiments, applying an epidermal cellular solution to the bio-absorbable graft can improve healing and prevent hernia recurrence.

In further embodiments, the graft material can further comprise therapeutics and/or drugs, such as for the sustained or controlled release of such therapeutics and/or drugs. Such agents can be used to prevent and/or treat progression and/or symptoms of disease (such as those diseases and symptoms described herein), and can also be used to prevent, treat, and or alleviate unwanted side effects of graft implantation. Non-limiting examples of unwanted side effects of implantation or grafting, for example, comprise pain, infection, inflammation, or scarring. Such unwanted side effects can be prevented, treated, or relieved through sustained, controlled, local release of drugs and/or therapeutic agents from the polymer or the graft material. For example, the addition of at least one anti-biotic, at least one anti-inflammatory, and/or at least one analgesic and/or anesthetic could prevent infection, reduce local inflammation and decrease pain at the surgical and/or implantation site, thus, for example, providing symptomatic relief.

The graft material can be mixed with therapeutic and/or prophylactic agents allowing for sustained release of the therapeutic and or prophylactic agent. Non-limiting examples of such agents comprise antibiotics, pain relievers, anti-inflammatories, or any combination thereof.

“Antibiotic” can refer to an agent that controls the growth of bacteria, fungi, or similar microorganisms, wherein the substance can be a natural substance produced by bacteria or fungi, or a chemically/biochemically synthesized substance (which may be an analog of a natural substance), or a chemically modified form of a natural substance. One of skill will recognize that the scaffold can be coated with a wide variety of antibiotics, such as penicillin, cephalosporins, macrolides, fluoroquinolones, sulfonamides, tetracyclines, aminoglycosides, and the like.

“Pain reliever” can refer to an agent that can provide relief from pain. An analgesic is any member of a group of drugs used to achieve analgesia, i.e., relief from pain. For example, the analgesic can be a pyrazolone derivative, such as (ampyrone, dipyrone, antipyrine, aminopyrine, and propyphenazone), aspirin, paracetamol, a non-steroidal anti-inflammatory (such as Ibuprofen, diclofenac sodium, or naproxen sodium), an opioid (such as codeine phosphate, tramadol hydrochloride, morphine sulphate, oxycodone), or any combination thereof. An anesthetic refers to any member of a group of drugs used to induce anesthesia—in other words, to result in a temporary loss of sensation or awareness of pain. Non-limiting examples of anesthetics comprise benzocaine, chloroprocaine, cocaine, cyclomethycaine, dimethocaine, larocaine, piperocaine, propoxycaine, procaine, novocaine, proparacaine, tetracaine, amethocaine, articaine, bupivacaine, cinchocaine, dibucaine, etidocaine, levobupivacaine, lidocaine, lignocaine, mepivacaine, prilocaine, ropivacaine, trimecaine.

An “anti-inflammatory” refers to a substance that treats or reduces the severity of inflammation and/or swelling. Non-limiting examples of anti-inflammatories comprise steroidal anti-inflammatories (such as corticosteroids) and non-steroidal anti-inflammatories (such as aspirin, celecoxib, diclofenac, diflunisal, ibuprofen, indomethacin, ketoprofen, ketorolac, nabumetone, naproxen, oxaprozin, piroxicam, salsalate, sulindac, tolmetin).

Sustained-release grafts have a common goal of improving treatment and/or symptomatic relief over that achieved by their non-controlled counterparts. The use of an optimally designed sustained-release preparation in medical treatment can be characterized by a minimum of drug substance being employed to cure, control, and/or provide relief of the condition in a minimum amount of time. For example, the sustained-release grafts can release an amount of a drug over the course of 1 day, 1 week, 2 weeks, 3 weeks, 4 weeks, 2 months, 3 months, 4 months, 5 months, 6 months, 7 months, 8 months, 9 months, 10 months, 11 months, 12 months, or longer. Advantages of sustained-release formulations include extended activity of the drug, reduced dosage frequency, and increased patient compliance. In addition, sustained-release formulations can be used to affect the time of onset of action or other characteristics, such as blood levels of the drug, and can thus affect the occurrence of side (e.g., adverse) effects.

Most sustained-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released at a rate that will replace the amount of drug being metabolized and excreted from the body. Sustained-release of an active ingredient can be stimulated by various conditions including, but not limited to, pH, temperature, enzymes, water, or other physiological conditions or compounds.

The graft material can comprise growth factors, such as endogenous growth factors, cytokines, chemokines, and protease inhibitors, many of which function to stimulate paracrine responses in fibroblasts, endothelial cells, and stem cells to promote tissue healing and repair. Previous studies have identified over 226 growth factors, cytokines, chemokines, and protease inhibitors, many of which function to stimulate paracrine responses in fibroblasts, endothelial cells, and stem cells to promote tissue healing and repair. In addition, these bioactive factors, including epidermal growth factor (EGF), fibroblast growth factor-4 (FGF-4), and TGF-β1 are known to promote proliferation, migration, and secretion of paracrine factors by fibroblasts, endothelial cells, and a variety of adult stem cells, including bone marrow-derived mesenchymal stem cells, adipose-derived stem cells and hematopoietic stem cells. The graft material can be formulated for increased hematopoietic stem cell recruitment and enhanced angiogenesis. In such embodiments, the graft material can comprise active growth factors and other biomolecules that retain the ability to direct or supplement biological activity, for example, by regulating endogenous cells in a wound environment. Non-limiting examples of such growth factors such as EGF, FGF-4, and TGF-β1.

Methods of Treatment

Aspects of the invention are directed towards methods of implanting a graft material in a subject to promote fascial healing, prevent hernia development, prevent hernia recurrence, or prevent the failure of hernia repair. Such aspects are applicable to subjects who are suffering from a hernia and subjects who are at risk of developing a hernia.

The term “subject” or “patient” can refer to any organism to which aspects of the invention can be administered, e.g., for experimental, diagnostic, prophylactic, and/or therapeutic purposes. Typical subjects to which compounds of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to a subject noted above or another organism that is alive. The term “living subject” refers to the entire subject or organism and not just a part excised (e.g., a liver or other organ) from the living subject.

Subjects at risk of developing a hernia refers to a subject who has a significantly greater risk of developing a hernia than the average risk of an age-, and sex-matched individual from the general population. See, for example, Ahn, Byung-Kwon. “Risk Factors for Incisional Hernia and Parastomal Hernia after Colorectal Surgery.” Journal of the Korean Society of Coloproctology 28.6 (2012): 280, which is incorporated by reference herein in its entirety.

For example, subjects at risk of developing a hernia comprise those who are obese, afflicted with an infection (such as a surgical site infection, intra-abdominal infection, deep tissue infection, or superficial abdominal infection), underwent a bowel resection, underwent colon surgery, are being administered corticosteroids, smokes, has chronic obstructive pulmonary disease, malnutrition, diabetes, immunosuppression, anemia, hypoproteinemia, male gender, old age, increased abdominal pressure (such as coughing, vomiting, distention, and ascites), or any combination. For example, subjects at risk of developing a hernia include those who are undergoing surgical incision of the abdominal wall, subject who are born with a congenital hernia, and/or subject who participate in activities that predisposes them to hernias, such as sports hernias or excessive coughing.

In the case or incisional hernias, the methods described herein can be performed during the procedure which requires the incision. In the case of procedures which require incision in an abdominal wall, the methods described herein can be performed after the incision is made. The methods can be performed before the incision is closed or after the incision is closed.

In an alternate aspect, the invention is directed to a method of repairing a hernia. In various embodiments, the methods described herein can be used to repair any type of hernia known to one of skill in the art. Exemplary hernias include incisional hernias, ventral hernias, umbilical hernias, epigastric hernias, lumbar hernias, inguinal hernias, diaphragmatic hernias, hiatal hernias, Spigelian hernias, or parastomal hernias.

The graft material can be aligned with the opening in the abdominal wall. This is particularly suitable when the graft material is provided as a sheet or sheet-like composition. For example, the graft material is generally placed or arranged in a position so to generally mirror the opening, such as the surgical incision. Thus, the graft material can promote healing of the surgical incision, such as healing of the abdominal fascial edges.

The graft material does not necessarily need to be actively affixed to the tissue to promote healing, but instead can simply be placed as an overlay or underlay in contact with or in close proximity to the opening. For example, the graft material can be implanted over or ventral to an abdominal wall opening, and thus promote healing of the opening. In other embodiments, the graft material can be implanted superficial to an abdominal wall opening, and thus promote healing of the opening. In a particular embodiment dermal graft is placed directly on top of (overlay) an abdominal fascia incision that has been closed with suture, and the dermal graft is not secured. The subcutaneous fat and skin are then closed over the dermal graft.

In alternative embodiments, the graft material or portion thereof can be anchored to the opening, such as an opening in the abdominal wall. The graft material can be anchored to the tissue opening by fasteners known to the skilled artisan, such as by pressure, an adhesive (such as fibrin glue), a clip, a tack, a suture, a staple, or a screw. For example, the graft tissue is implanted under, deep to, or dorsal to the abdominal wall opening, such as by sutures.

In embodiments, the opening, such as the abdominal wall opening, can be substantially or completely closed prior to implanting of the graft material. In a particular embodiment, dermal graft is secured with sutures, staples, screws, or other securement devices under (underlay or sublay) an abdominal fascia incision that has been closed with suture. The subcutaneous fat and skin are then closed over the incision.

In certain embodiments, a cellular suspension, as described herein and further described in U.S. Pat. Nos. 9,029,140 and 9,078,741, can be applied to the abdominal wall opening or other graft site. The cellular suspension can be sprayed through any type of nozzle that transforms liquid into small airborne droplets. In embodiments, appropriate nozzles and spraying conditions avoid subjecting the cells in solution to shearing forces or pressures that would damage or kill substantial numbers of cells. Certain embodiments employ spraying methods that do not require that the cellular suspension to be mixed with a propellant fluid that is toxic or detrimental to cells or wound beds. A variety of appropriate nozzles ad spraying methods are commonly available. Such nozzles may be connected in any conventional way to a reservoir that contains the cellular suspension.

Alternatively, the suspension can be delivered via a pipette, common “eye-droppers. Syringe and needle and or other similar devices to place Small quantities of cellular suspension on a graft site.

After the cell suspension has been applied to the recipient graft site, the wound can be dressed with a wound dressing. In one embodiment the dressing comprises a woven nylon dressing.

Surgical mesh, such as a synthetic mesh or biological mesh, is a currently available tool in hernia repair; however, are fraught with postoperative complications. Common complications include infection, pain, adhesions, seroma mesh extrusion and hernia recurrence. Reducing the complications of mesh implantation is of utmost importance given that hernias occur in hundreds of thousands of patients per year in the United States. Thus, in an embodiment, the opening is closed with a surgical mesh, and the graft material is placed or affixed over the surgical mesh or affixed to the undersurface (“underlay”) of the surgical mesh to promote healing of the tissue and reduce unwanted side effects. In a particular embodiment, a hernia defect is closed with synthetic mesh, and the dermal graft (either as intact sheets, micronized form, or powder form, or a combination thereof) is placed on top of areas where the mesh interfaces with the fascia, or under areas where the mesh interfaces with the fascia, or under areas where the mesh interfaces with the abdominal cavity and its contents (e.g. large/small intestine/other intraabdominal organs such as liver, stomach, spleen). The dermal graft may or may not be secured with sutures, staples, screws, or other securement device. The subcutaneous fat and skin are then closed over the dermal graft.

In embodiments, the epidermis can be removed from a dermal graft, such as by chemical means or enzymatic means. The cellular elements of the epidermis can then be used to assist with hernia repair, such as in a spray form. For example, the cellular elements can be sprayed onto a biologic or synthetic mesh to improve healing, promote incorporation, and cover the mesh with living cells to decrease infection. In another example, the cellular elements can be sprayed onto an orthopedic implant, such as to improve healing, promote incorporation, and/or cover the implant with living cells to decrease infection.

In any of the various exemplary embodiments disclosed herein, the dermal graft, such as that created via mechanical or enzymatic debridement, comprises a tensile strength that is substantially equivalent the tensile strength of a full thickness skin graft. The dermal graft can comprise a tensile strength as low as about 10 N/cm. In embodiments, the tensile strength of the dermal graft can be as high as 1500 N/cm. The tensile strength of the dermal graft can be between about 25 N/cm to about 1000 N/cm. In certain embodiments, the tensile strength of the dermal graft is between about 50 N/cm to about 800 N/cm. In embodiments, the dermal graft comprises a tensile strength of up to about 700 N/cm. In one embodiment, the dermal graft comprises a tensile strength of about 600 N/cm.

In other embodiments, the opening, such as the abdominal wall opening, can be substantially or completely closed after implanting the graft material. For example, the graft material can be placed or affixed under the opening, and then the opening can be substantially or completely closed, thereby allowing the graft material to promote healing of the tissue.

The breakdown of a hernia repair is called recurrent hernia. The bulge returns at or near the site of the prior hernia. Recurrent hernias greatly increase the complexity of subsequent repair. If left untreated, severe complications can result such as the intestines being trapped known as an incarcerated hernia, digestive obstruction, or a loss of blood supply to the intestines known as a strangulated hernia. Thus, aspects of the invention are directed towards a method of preventing hernia recurrence.

In one aspect, the invention is directed to preventing the formation of one or more cysts or seromas following implantation of a graft material. In certain embodiments, the graft material is substantially free of epidermal cells before implantation. Embodiments include removal of the epidermis or removal of substantially all epidermal cells from the graft material. In embodiments, epidermal inclusion cyst formation is reduced following implantation of a skin or cutis graft.

In various aspects, the invention is directed to methods of promoting facial healing, methods of preventing hernia recurrence, methods of treating a medical condition with a regenerative cell suspension.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Introduction: Hernia repair is the most common procedure performed with over one million surgeries in the United States each year. Synthetic mesh is used in over 80% of hernia repairs with 12% of patients developing a subsequent infection and complication involving the mesh. These complications have led to complex, severe injuries, dramatically impacting patients' lives and over $1 billion in settlements in the United States. In 2006 3.2 billion was spent on abdominal hernia repairs in the US alone. As an alternative, autologous skin can be taken from the patient during the hernia repair with data from over 100 patients at Louisiana State University Health Science Center and University Medical Center showing a reduction in early complications over 11 months. Long term complications such as recurrence are still being studied in this population while prior data from Europe demonstrated comparable long term outcomes to synthetic mesh techniques. The most impressive advantages of autologous skin use for hernia repair include:
1. Reduced inflammation due to lack of foreign body and availability for engraftment
2. Use of native tissue as opposed to expensive synthetic or bioprocessed tissues/mesh
3. Able to be used in contaminated fields and high-risk patients
4. Less pain relative to synthetic mesh
5. Mitigation of litigation for a common procedure

The key components of processing autologous dermal grafts for hernia repair includes harvesting the full-thickness autograft, perforating the graft for efflux of serous fluid, and removal of the graft epidermis. Current methods of full-thickness autograft harvest are performed with sharp excision or electrocautery sparing native adipose tissue. Autograft methods can be very time intensive. New meshing devices are being investigated to easy graft perforation.

The most challenging part of the surgery is the removal of epidermis to prevent seroma or epidermal cyst formation. This step is currently performed mechanically with an electrocautery scratch pad or blade that may lead to graft damage and requires 20 minutes on average of dedicated surgeon time for a 100 cm² graft. The ability of the RECELL® device to disaggregate the epidermis could greatly improve surgeon acceptance of the technic due to decreased operating room time, and offers the potential to reduce long term complications due to the relatively high proportion of fibroblasts in the autologous skin cell suspension.

Without wishing to be bound by theory, enzymatically processed full-thickness grafts are equivalent or superior to non-enzymatically processed in hernia repair and an autologous skin cell suspension can be utilized in engraftment of the hernia repair.

Without being bound by theory, the RECELL® enzyme system can be used to process full thickness skin grafts to be used in hernia repair. Further, the epidermal solution can be applied back onto the wound to increase healing, or the enzyme can simply be used to remove epidermis to prevent epidermal inclusion cyst formation in a hernia repaired with a cutis graft. If applied back onto the wound to increase healing, the epidermal solution, which contains living, viable cells, can promote tissue growth and tissue healing, such as by releasing growth factors that promote tissue growth and tissue healing.

### Example 2

Description of the Technology:

1. Use of RECELL® enzyme for deepithelialization of autologous tissue in hernia repair.

2. Use of RECELL® enzyme to obtain regenerative cell suspension for use in chronic wounds.

3. Use of RECELL® enzyme to obtain regenerative cell suspension for use in exfoliative skin diseases.

4. Use of RECELL® enzyme to obtain regenerative cell suspension for use in surgical wound closure.

5. Use of RECELL® enzyme with autologous tissue and dermal regeneration template for definitive wound closure.

Hernia repair and chronic wounds are two of the biggest markets for medical devices in the world with billions of dollars spent yearly on their treatment. Any commercial product which can convert a patient's own skin to a material which can repair or prevent hernia, and also heal chronic wounds would be extremely profitable.

There are no current products on the market to process a person's own skin into a hernia mesh, or a treatment for chronic wounds. RECELL® has shown to regrown human epidermis.

Without wishing to be bound by theory, RECELL® can be used to produce strong full thickness skin after treatment with the enzyme in human/animal, and animal/human studies to promote wound healing.

### Example 3

### The Use of Autologous Fenestrated Cutis Grafts in Hernia Repair: Surgical Outcome and Cost Analysis of 97 Consecutive Patients

Introduction

Hernia repairs (HRs) are among the most common operations performed by surgeons worldwide. In the United States alone, approximately 300,000 and 700,000 operations are performed annually for ventral and inguinal hernias, respectively (1). Incisional hernia (IH) repair costs are between 6 to 10 billion USD per year (2). Various techniques for inguinal and ventral hernia repairs exist in the literature. In most procedures, the use of synthetic mesh is the standard technique to reinforce the repair because of its significantly lower hernia recurrence rates compared to primary suture repair (3). Recurrence rates associated with primary suture repair can approach 54%, whereas the highest mesh repair rates are 36% (4). Mesh repair, however, is associated with significant morbidity and cost. The use of non-absorbable mesh can lead to seroma, fistula, infection, pain, mesh fracture, exposed mesh, hernia recurrence from shrinkage, and chronic foreign body reactions (5). Synthetic mesh is usually less expensive than biologic prostheses, but a synthetic mesh infection can add an additional 108,000 USD in annual charges (6). Biologic implants have been suggested as an alternative, especially in infected surgical fields; at 25-30 USD per square cm, however, their cost can be prohibitive (7). As such, HR currently lacks an effective, reliable, safe and cost-efficient solution for fascial reinforcement.

Without wishing to be bound by theory, an alternative method of HR can use full-thickness cutis autografts (CA). The use of CA predates the advent of synthetic mesh products. The original HR technique as described by Loewe in 1913 consisted of a full-thickness skin graft, which was denuded of epidermis with the use of a knife, and was subsequently used successfully as an overlay to bolster a primary HR (8). CA have the advantage of avoiding some of the morbidities associated with synthetic mesh and the high cost of biologic mesh. We performed a multi-surgeon, multi-institutional retrospective study of the use of CA for open and laparoscopic HRs in 97 patients over a 10.50-month follow-up period to determine its overall complication and recurrence rate.

Materials and Methods

This study was conducted at an academic teaching hospital and an urban community hospital by 5 different surgeons. Patients with abdominal wall hernias were offered the opportunity to undergo HR using mesh or CA. Patients with inguinal hernias were only offered CA if they had very high-risk factors, were undergoing removal of an infected prosthesis, or were adamant about not having mesh placed. After hospital discharge, patients underwent routine follow up with the operating surgeons. Data were collected on the first 99 consecutive patients in which CA were used. No exclusion criteria existed. Demographic data and complications were compiled on each patient. Endpoints measured included all post-operative complications including hospital readmission, surgical site infection (SSI), SSI requiring intravenous (IV) antibiotics, superficial skin dehiscence, seroma, skin flap necrosis, mesh removal/explant, hernia recurrence, reoperations, enteric fistula formation, and bowel obstruction.

Inguinal hernias were repaired using the conventional open Lichtenstein technique, with use of CA in lieu of mesh, which were sutured with slowly absorbable suture. Abdominal wall hernias (incisional, epigastric, umbilical, and lumbar hernias) were repaired using a variety of laparoscopic or open techniques based on the characteristics of the defect and surgeon preference. The abdominal wall fascia was closed with absorbable suture in all cases. Most often, CA was implanted in an underlay position, although sublay retro-rectus, sublay extra-peritoneal, and onlay placements were also used. Ten hernias were repaired laparoscopically. In these CA, the hernia defect was closed by way of a trans-fascial suture passer, and then the CA was fixed as an underlay implant with trans-fascial absorbable sutures and absorbable laparoscopic tacks.

CA were prepared by harvesting full-thickness skin grafts. The location and shape of such grafts were based on hernia size and anatomic location: groin for laparoscopic repairs, pannus for obese patients, or skin near the hernia defect. In general, grafts were elliptical in shape. For smaller umbilical hernia repairs, however, a crescent shaped area of skin was harvested near the umbilicus. All donor sites were closed primarily without excess tension. Preparation of the full-thickness skin graft was performed on the back table by first removing any excess adipose tissue from the dermis. The epithelium was removed by mechanical debridement with an electrocautery scratch pad, pineapple burr, or Norsen debrider. The skin was then perforated in a “pie-crust” fashion with an 11-blade or 16-gauge needle, thereby providing a perforated cutis graft. The graft was then placed within a solution of 50% normal saline and 50% hydrogen peroxide 3% solution to remove tissue debris and prevent potential contamination. For graft placement, the dermal side was placed against the fascia. For onlays, the dermis was placed down against the fascia with the epidermal side facing the adipose tissue. With underlay repairs, the dermal side was placed against the fascia, with the epidermal side facing posterior toward the abdominal contents. When the graft was placed in the retro-rectus space, the dermis was affixed to the rectus muscle.

For a cost-comparison analysis, we obtained a list of all mesh charges assigned to 2 of our main surgeons for abdominal wall hernias (incisional, umbilical, lumbar, epigastric) performed at the academic teaching hospital for identical periods of the year. Hospital costs related to the mesh used were then calculated and the annual cost extrapolated to 12 months.

Results

Patient Demographics

In this study, 97 patients underwent HR with CA. Patient demographics are summarized in Table 1. Approximately half of the patient population were male (52%). The overall average age was 67.18 years old. The overall average BMI was 33.09 (Class 1 Obesity).

65% of patients had a prior abdominal procedure, and 36% of patients had a history of prior HR. 10 patients were immunocompromised, of which 6% had human immunodeficiency virus (HIV). A significant number of our patient population had documented chronic medical conditions: 17% of patients were diabetic and 53% had hypertension. 19% of patients were active tobacco users, whereas 35% were former tobacco users.

Operative Details

Operative characteristics are summarized in Table 2. Open techniques were used for 89 of the repairs/placement, and the remaining ten were performed using a laparoscopic approach. There were seven inguinal hernias that repaired. The majority of ventral hernias were incisional (44) and umbilical (34). Other defects included including epigastric (nine), spigelian/lumbar/femoral (five) hernias.

Most of the hernias were repaired using a CA in the underlay position (58), whereas 19 were repaired using an onlay and 5 in a retrorectus position. A sandwich technique (onlay plus an underlay or sublay) was used for nine of the repairs. In eight instances, documentation of type of hernia could not be found. The average CA size was 107.2 cm2.

Wound Complications and Patient Outcomes

Postoperative complications are reported in Table 3. Our average length of follow-up after surgery was 10.50 months, and these records were available for 95 patients. Nineteen patients had complications. Three patients had a postoperative hematoma. Nine patients had surgical site infections: six of these patients required intravenous antibiotics. Two patients presented with a postoperative seroma requiring evacuation. Superficial skin dehiscence occurred in 6 patients, and superficial skin necrosis was seen in 2 patients. Three patients had hernia recurrences. None of the patients developed bowel obstruction, enteric fistula formation or had CG removed. Three patients died during the follow-up period: one patient suffered a fatal postoperative myocardial infarction; the second patient developed a leak following a colostomy take down with subsequent demise; the third patient died after being struck by a motor vehicle as a pedestrian.

Cost Analysis

The average monthly mesh cost for the last 10 months was 19,013 USD, and this number dropped to 7,465 USD for the last 10 months of the following year. The extrapolated annual average monthly cost for mesh for 2 surgeons performing HRs at the academic teaching hospital (84% of HRs performed by these surgeons) was 228,153 USD. After adoption of the CA technique, this number dropped to 89,475 USD in the following year, a 61% reduction in mesh cost. This decrease resulted in a yearly savings of 138,568 USD. The true savings is likely higher due to the fact that we changed our technique at our secondary hospital where we perform 16% of our hernia repairs.

In consultation with local insurance payers, the hospital billing department, and our group practice billing company, we use CPT codes of 15200 for full-thickness skin graft up to 20 square centimeters, and 15201 for placement of full-thickness skin graft for each additional 20 square centimeters when placing a perforated cutis graft. Using the calculation of 33 USD per square cm for biologic mesh, and the Medicare reimbursement of 37.89 USD per Relative Value Units (RVU) as a comparison, the CPT code 15200 reimburses 9.15 RVU, and the CPT code 15201 reimburses 1.32 RVUs. This translates to savings of 319 dollars for the first 20 cm of the CA graft, and then increases to savings of 610 USD for each additional CA graft size. The use of cutis grafts in place of biologic and synthetic mesh will shift to a higher RVU reimbursement to the surgeon, with less overall cost to patients, hospitals, and insurance providers. The two surgeons with complete 2017 and 2018 data were not able to obtain a report of how much of an RVU increase this new technique generated due to a hospital software change, but they have seen an increase in total RVU in 2018 over the previous year.

Discussion

Almost one million ventral and inguinal operations are performed annually in the United States, costing the national healthcare system up to 10 billion USD per year. Currently, there are over 200 different brands of synthetic and biologic prostheses are on the market in the United States. The plethora of implant options emphasizes that the ideal prosthesis has yet to arrive for HR. Such an implant should be chemically inert and resistant to mechanical strain and infection, amenable to tailoring its size, and the ability to be sterilized, prevent visceral adhesions, and behave similar to autologous tissue (9). Additionally, it should be low-cost and enable tissue ingrowth.

CA satisfies all these criteria. It has the same tensile strength as biologic prostheses yet it remains soft enough to be placed against organs (10). For example, dermis harvested during pannieculectomy procedures demonstrate a tensile strength of 15.9 Mpa (average maximum load before yielding 680), similar to the FlexHD biological graft average tensile strength of 15.7 Mpa, (average maximum load of 69.8 prior to yielding) (11). CA also show rapid ingrowth of host tissue and develop patent vascular ingrowth within 48 hours (12). Finally, it is low-cost. Mesh prices range from 40 USD to 40,000 USD depending on the material and its coating. In general, synthetic mesh is lower cost, but it carries a higher risk of infection, adhesions, and fistula (13). Biologic prostheses are more expensive with a lower risk of infection and fistula, and may be utilized in contaminated surgical fields (14). CA therefore present an opportunity to decrease costs by using a “free” prosthesis while also minimizing the risks of infection associated with synthetic mesh (15).

Autologous hernia repair is not a new concept. Since Loewe first proposed the use of dermal grafts for hernia repair (8), this method has been widely explored by a variety of authors. By 1929 Loewe, himself, performed cutis grafts in about 100 patients. Whole skin graft, dermal only grafts and strips of graft have all been documented in previous literature. Authors reported acceptable results with this method; however, complications such as sinus tracts, cyst formation, epidermoid carcinoma and squamous cell carcinoma were also reported. Use of this method may have dwindled after reports like that of Peterson and Ludwig further warned surgeons against using this method due to previous reports of malignancy associated with cutis grafts. In our review of the literature there are only two reports of cutis graft associated carcinomas. Albeit, epidermoid cyst transformation into squamous and basal carcinomas have been. Overall, the risk of squamous cell carcinomas associated with epidermoid cysts is low: 0.011 to 0.045% and the association between cutis grafts and carcinoma has not been proven. Other case reports document carcinoma associated with other methods such as synthetic mesh repairs. Notably, dermal grafts are currently used for a variety of reconstructive procedures.

Contemporary international reports have demonstrated the safe use of CA in HR, noting that such CA, for example, only use full thickness skin grafts, did not perforate the skin grafts, and only placed as an onlay. For example, Clay et. Al (16) performed a multicenter, randomized study with 24 patients comparing sublay or onlay synthetic mesh to onlay autologous dermal graft in HR. Average follow-up time was 3 months, and all patients underwent evaluation for hernia recurrence by physical exam. No significant difference was present between the two groups related to seroma formation and surgical site infection rates. Recurrence rates were the same for each group (4.17%). Patients treated with synthetic mesh more frequently reported pain at the surgical site at 3-months post-op compared to the CA group (16). Literature to date reports varied rates of post-operative complications: 2.7-25% for SSI, 2.7-12.5% for seroma, and 0-25% for hernia recurrence (11,17-21).

Our surgical technique was developed to enhance CA incorporation and minimize complications. To minimize the risk of epidermal inclusion cyst formation, we removed as much of the epidermis as possible (22-24). This removal also includes the nonviable stratum corneum layer, helping facilitate in-growth. To decrease the rate of subgraft seroma/hematoma formation, we perforate the CA to allow fluid to pass to either side of the graft. Perforation of cutis grafts for hernia is unique. In response to research demonstrating asymmetric patterns and density of collagen, we place the dermal side of the graft toward the tissue being reinforced. The deeper layer of dermis, where vessels and nerves penetrate, has looser connective tissue that facilitates faster tissue ingrowth. In contrast, the connective tissue becomes denser as it approaches the epidermis, becoming more resistant to tissue in-growth. Finally, we close the fascial defect during HRs (except for one CA) to eliminate the higher hernia recurrence rates associated with bridging repairs (25). Thus, this study improves upon previously developed methods using dermal grafts for HR. Unlike previous techniques, however, we do place CGs in the retrorectus space or as an underlay implant, we perforate our CA to prevent subgraft seroma, and we have employed laparoscopic techniques to place CA.

Our rate of complications using the CA approach for HR is similar to that published in the literature (15,26). In the majority of CA, these complications involved superficial wound infections, seromas, and hematomas, which were treated with local wound care in clinic, interventional radiology drainage, or incision and drainage in the operating room. Three hernia recurrences occurred during the follow-up period. The first CA involved a low supra-pubic incisional hernia repaired laparoscopically. Two months post-procedure, the patient suffered a fall onto her abdomen and reported feeling a “pop.” Computed tomography (CT) of the abdomen/pelvis confirmed the presence of a recurrent hernia with bowel within the left labia. On re-operation, the graft had separated from the pubic bone. Repair involved performing an open retrorectus procedure with placement of another CA in this space. A biopsy of the incorporated sample from her first graft underwent pathologic evaluation, and the results showed no cyst or hair formation. In addition, no skin elements were present on the sample, representing complete incorporation of the graft into the fascia. This tissue contained macrophages and had the microscopic appearance of connective tissue. In the second CA, the CA sutures had pulled off from the sidewall following an open umbilical repair. The third recurrence became apparent after a CT, which demonstrated that the fascial closure had reopened. The onlay CA, however, remained intact covering the defect. Given the fact that the patient did not have any pain or palpable defect, this finding was not clinically relevant, and, consequently, it was not repaired.

Unlike prior reports related to acellular human cadaveric grafts used in HR (27, 28), we did not encounter any weakening or eventration of our CA during the follow-up period. In addition, we did not encounter the development of any enteric fistulas, and we did not have to re-operate for bowel obstruction due to adhesions. Importantly, CA did not require explant in the setting of infection. Examination of the CA in which wounds required re-exploration revealed that it remained viable and resistant to degradation. Consequently, healthy granulation tissue developed over the CA with negative pressure dressings or packing. Also, when the grafts were left exposed to the atmosphere to heal by secondary intention, they did not undergo the degradation of an exposed or infected biologic graft, and all the wounds healed well without sinus tracks. Such observations indicate that this technique can be durable in the setting of extremely high-risk wounds, making treatment of infections around or near prostheses much more straightforward. When synthetic mesh becomes infected, a successful non-operative treatment rarely works. Additionally, the antigenic properties of biologic prostheses can lead to its degradation in infected fields.

In summary, this study validates that CA use is safe with comparable outcomes to HR using other meshes on the market. It seems to perform well in obese, high-risk patients, and in infected fields. In our modern social setting of heightened fear of any mesh complications, despite discussion of the risk of epidermal inclusion cysts, patients invariably opt for CA HR after discussing the risks and benefits of synthetic, biologic, and dermal autograft prostheses with them. Another benefit of the CA is its low cost compared to other mesh products. Since it comes from the patient's own body, it is a particularly attractive option in HR in underdeveloped nations where even the least expensive mesh is out of reach for patients due to extreme poverty (29). Benefits of the CA extend beyond those for the patient to include societal advantages including a decreased reliance on the harvesting of xerographic biologic prostheses from animals, elimination of conflicts with religious beliefs and the use of animal products, decreased petroleum usage to create most synthetic meshes, and the elimination of the need for cadaveric harvesting of tissue.

### REFERENCES CITED IN THIS EXAMPLE

Kuwada T. The Management of Inguinal Hernia. In Cameron J L, Cameron A M, ed. Current Surgical Therapy, 12th Ed. Philadelphia, Pa.: Elsevier; 2017: 623-628.
Allied Analytics LLP. Hernia Repair Devices and Consumables Market by Product, Surgery Type, and Hernia Type—Global Opportunity Analysis and Industry Forecast, 2016-2023. Available at: https://www.alliedmarketresearch.com/hernia-repair-devices-market. Accessed 16 Feb. 2017.
Payne R, Aldwinckle J, Ward S. Meta-analysis of randomised trials comparing the use of prophylactic mesh to standard midline closure in the reduction of incisional herniae. Hernia. 2017 December; 21(6):843-853. PubMed PMID: 28864937.
Sanders D L, Kingsnorth A N. The modern management of incisional hernias. BMJ. 2012; 344:e2843.
Goodenough C, Ko T C, Kao L S, et al. Development and Validation of a Risk Stratification Score for Ventral Incisional Hernia after Abdominal Surgery: Hernia Expectation Rates in Intra-Abdominal Surgery (The HERNIA Project). J Am Coll Surg. 2015 April; 220(4):405-13. doi: 10.1016/j.jamcollsurg.2014.12.027. Epub 2015 Jan. 2.
Augenstein V, Colavita P, Wormer B, et al. CeDAR: Carolinas Equation for Determining Associated Risks. JACS. October 2015. 221(4): S65-S66.
Petro C C, Rosen M J. A Current Review of Long-Acting Resorbable Meshes in Abdominal Wall Reconstruction. Plast Reconstr Surg. 2018; 142(3 Suppl):84S-91S.
Loewe I. Uber Haut-Tiefenplastik. 1925. Munch Med Wochenschr. 76:2125.
Shankaran V, Weber D J, Reed R L, Luchette F A. A review of available prosthetics for ventral hernia repair. Ann Surg. 2011; 253(1):16-26.
Gallagher A J, Anniadh A N, Bruyere K, et al. Dynamic Tensile Properties of Human Skin. Paper presented at: International Research Council on Biomechanics of Injury Conference; 2012; Dublin, Ireland.
Özkaya mutlu Ö, Egemen O, Akan A, et al. The use of dermal automesh for incidental hernia repair in abdominoplasty: Clinical, biochemical, and radiological results. J Plast Surg Hand Surg. 2015 June; 49(3):172-6. doi: 10.3109/2000656X.2014.976571. Epub 2014 Nov. 11.
Thornton J. Skin Grafts and Skin Substitutes and Principles of Flaps. In Thornton J, Gosman A A., ed. Selected Readings in Plastic Surgery, Vol. 10, No. 1. Dallas, Tex.: Selected Readings in Plastic Surgery: 2-23.
Odd L., Kristoffersen A., Oral, intestinal, and skin bacteria in ventral hernia mesh implants, J Oral Microbiol. 2016; 8: 10.3402/jom.v8.31854. Published online 2016 Jul. 29. doi:10.3402/jom.v8.31854
Muysoms F E, Jairam A, López-Cano M, Śmietański M, Woeste G, et al. Prevention of Incisional Hernias with Biological Mesh: A Systematic Review of the Literature. Front Surg. 2016. 3(53). PubMed PMID: 27725931; PubMed Central PMCID: PMC5035749.
Reynolds D, Davenport D L, Korosec R L, Roth J S. Financial implications of ventral hernia repair: a hospital cost analysis. J Gastrointest Surg. 2013 January; 17(1):159-66; discussion p. 166-7. doi: 10.1007/s11605-012-1999-y. Epub 2012 Sep. 11.
Clay L, Stark B, Gunnarson U, Strigard K. Full thickness skin graft vs. synthetic mesh in the repair of giant incisional hernia: a randomized controlled multicenter study. Hernia. 2018 April; 22(2):325-332. doi: 10.1007/s10029-017-1712-x. Epub 2017 Dec. 15.
Korenkov M, Sauerland S, Arndt M, Bograd L, Neugebauer E A, Troidl H. Randomized clinical trial of suture repair, polypropylene mesh or autodermal hernioplasty for incisional hernia. Br J Surg. 2002; 89(1):50-6.
Martis G, Damjanovich L. Use of double-layer autologous dermal flap in the treatment of recurrent and/or infected incisional hernias: presentation of the surgical technique and the results of 1-year follow-up-a prospective, consecutive cohort study. Hernia. 2016; 20(3):461-70.
Strahan A W B. Hernial Repair by whole-skin graft, with report on 413 Cases. Br J Surg. 1951 January; 38(151):276-84.
Samson T D, Buchel E W, Garvey P B. Repair of infected abdominal wall hernias in obese patients using autologous dermal grafts for reinforcement. Plast Reconstr Surg. 2005; 116(2):523-7.
Earle A S, Abdel-fattah M A. Closure of an abdominal hernia with a groin flap lined with a dermal graft. Case report. Plast Reconstr Surg. 1975; 56(4):447-9.
Liou L S, Montague D K, Angermeier K W. Dermal graft repair of peyronie's disease complicated by epidermoid cyst. J Urol. 2003; 169(2):617-8.
Savoca G, Ciampalini S, De stefani S, Trombetta C, Belgrano E. Epidermoid cyst after dermal graft repair of Peyronie's disease. BJU Int. 1999; 84(9):1098-9.
Weinberg S, Kryshtalskyj B. Epidermoid cyst in a temporomandibular joint dermal graft: report of a Case and review of the literature. J Oral Maxillofac Surg. 1995; 53(3):330-2.
Holihan J L, Nguyen D H, Nguyen M T, et al. Mesh Location in Open Ventral Hernia Repair: A Systematic Review and Network Meta-analysis. World J Surg. 2016 January; 40(1):89-99. doi: 10.1007/s00268-015-3252-9.
Lindmark M, Strigård K, Löwenmark T, Dahlstrand U, Gunnarsson U. Risk Factors for Surgical Complications in Ventral Hernia Repair World. World J Surg. 2018 November; 42(11):3528-3536. doi: 10.1007/s00268-018-4642-6.
Blatnik J, Jin J, Rosen M. Abdominal hernia repair with bridging acellular dermal matrix—an expensive hernia sac. Am J Surg. 2008; 196(1):47-50.
Bluebond-langner R, Keifa E S, Mithani S, Bochicchio G V, Scalea T, Rodriguez E D. Recurrent abdominal laxity following interpositional human acellular dermal matrix. Ann Plast Surg. 2008; 60(1):76-80.
Yenli E M T, Abanga J, Tabiri S, et al. Our Experience with the Use of Low Cost Mesh in Tension-Free Inguinal Hernioplasty in Northern Ghana. Ghana Med J. 2017 June; 51(2):78-82.

TABLE 1
Demographics of Patients Undergoing Hernia Repair

| | N | % |
|---|---|---|
| Total Patients/Total Hernias | 97/99 | |
| Age | 67.18 | |
| Average Body Mass Index | 33.09 | |
| Male | 51 | 52% |
| History of Prior Hernias | 34 | 36% |
| Prior Abdominal Surgeries | 59 | 65% |
| Current Tobacco Use | 18 | 19% |
| History of Tobacco Use | 33 | 35% |
| Diabetes Mellitus | 16 | 17% |
| Hypertension | 50 | 53% |
| Coronary Artery Disease | 7 | 7% |
| HIV | 6 | 6% |

TABLE 2
Operative Characteristics of Patients Undergoing Hernia Repair

| | N | % |
|---|---|---|
| Open Repair | 89 | 90% |
| Laparoscopic Repair | 10 | 10% |
| Inguinal Hernia Repair | 7 | 7% |
| Ventral Hernia Repair | 92 | 94% |
| Incisional Hernia Repair | 44 | 48% |
| Umbilical Hernia Repair | 34 | 37% |
| Epigastric Hernia Repair | 9 | 10% |
| Other Hernia Repair (e.g. | 5 | 5% |
| Spigelian, Lumbar) | | |
| Onlay Graft Technique | 19 | 21% |
| Underlay Graft Technique | 58 | 64% |
| Retrorectus Graft Technique | 5 | 6% |
| Sandwich Graft Technique | 9 | 9% |
| Unreported Graft Technique | 8 | 8% |
| Average Full Thickness Skin | 107.2 | |
| Graft Size, cm² | | |

TABLE 3
Complications of Patients Undergoing Hernia Repair

| | N | % |
|---|---|---|
| Hospital readmission | 7 | 7% |
| Hematoma | 3 | 3% |
| Surgical Site Infection | 5 | 5% |
| Surgical Site Infection Requiring | 6 | 6% |
| Intravenous Antibiotics | | |
| Superficial Skin Dehiscence | 6 | 6% |
| Seroma | 2 | 2% |
| Skin Flap Necrosis | 2 | 2% |
| Mesh Removal | 0 | 0% |
| Hernia Recurrence | 3 | 3% |
| Any Reoperation | 7 | 7% |
| Enteric Fistula Formation | 0 | 0% |
| Bowel Obstruction | 0 | 0% |

### Example 4

Autologous Dermal Hernia Repair Study 31-001

Introduction

Hernia repair is the most common procedure performed with over one million surgeries in the United States each year. Synthetic mesh is used in over 80% of hernia repairs with 12% of patients developing a subsequent infection and complication involving the mesh. These complications have led to complex, severe injuries dramatically impacting patients' lives and over $1 billion in settlements in the United States. As an alternative, autologous skin taken from the patient during the hernia repair is an alternative with data from over 97 patients at Louisiana State University Health Science Center and University Medical Center showing a reduction in early complications. Long term complications such as recurrence are still being studied in this population while prior data from England demonstrated comparable long term outcomes to synthetic mesh techniques. The most impressive advantages of autologous skin use for hernia repair include:

Reduced inflammation due to lack of foreign body and availability for engraftment

Use of native tissue as opposed to expensive synthetic or bioprocessed tissues/mesh

Able to be used in contaminated fields and high-risk patients

Less pain relative to synthetic mesh

Mitigation of litigation for a common procedure

The greatest challenges to widespread adoption of this method is the absence of large study populations with long term data and intraoperative difficulty in processing a full-thickness autograft. The key components of processing autologous dermal grafts for hernia repair includes harvesting the full-thickness autograft, perforating the graft for efflux of serous fluid, and removal of the graft epidermis. Current methods of full-thickness autograft harvest are performed with sharp excision or electrocautery sparing native adipose tissue. New meshing devices are being investigated to easy graft perforation. The most challenging part remains the removal of epidermis to prevent seroma or cyst formation. This step is currently performed mechanically with an electrocautery scratch pad that may lead to graft damage and requires 20 minutes on average of dedicated surgeon time for a 100 cm2 graft. Without wishing to be bound by theory, the ability of the ReCell device to disaggregate the epidermis could greatly improve surgeon acceptance of the technic and can to reduce long term complications due to the relatively high proportion of fibroblasts in the autologous skin cell suspension. Our works will validate that the enzymatically processed full-thickness grafts are equivalent or superior to non-enzymatically processed in hernia repair and to validate the role of autologous skin cell suspension in engraftment of the hernia repair.

Objectives of this Study:
1. Use of a validated tensile strength model to compare the tensile strength of perforated mechanically created cutis graft, vs perforated ReCell created cutis graft.
2. Use of a validated rodent hernia model with full-thickness enzymatically treated cutis grafts specifically examining for evidence of early complications defined as evisceration, hernia occurrence, seroma, or infection.
3. Use of validated rodent model to examine engraftment of cutis grafts treated with autologous skin cell suspension relative to those not treated and tensile strength of 2×2 cm abdominal wall/graft sample.

Endpoints:
1. Rate of evisceration, hernia occurrence, seroma formation, and infection.
2. Rate of engraftment as determined using and tensile strength of 2×2 abdominal wall/graft sample.

Methodology:

A cutis graft is a procedure where the patient's own skin is harvested in a full thickness graft and epidermis and subcutaneous tissue removed. The graft can be sewn as an underlay underneath a laparotomy incision to prevent hernia (1). In addition to prevention, hundreds of patients underwent hernia repair with the Cutis graft in the early 1900s, but with Usher's use of synthetic graft repair of inguinal hernias, synthetic grafts became more common (2). In reviewing the literature, cutis mesh placed in the subcutaneous space or as a hernia repair loses its dermal structures and becomes a thick connective tissue. Peer and Paddock deeply implanted human dermal grafts and removed them for analysis at 1 week to 1 year. Their research showed that hair follicle and sebaceous glands were resorbed after 1 week, and sweat glands were in the process of degeneration in later analysis (3).

Our study will be to use expanded cutis graft as a novel mesh for prevention and treatment of hernias. The use of cutis graft has been used in the past in the United States and is currently being used in smaller studies in other countries for hernia repair. Cutis graft has the properties of an idea mesh, due to it not being a foreign material, sufficient strength to prevent hernia, resistance to infection, no cost, can be placed in contact with bowel or placed into infected fields.

This study will validate this novel technique for hernia prevention and treatment. Since there is a limit to the amount of skin which can be harvested for a cutis graft, and still close the wound primarily, meshing an ellipse of skin wound be ideal, if it can be shown that meshed skin will retain the necessary strength.

Our protocol will be to take fresh human dermis and freeze it for storage, or transport fresh to lab. The specimens will then be brought to room temperature if frozen. The epidermis will be sharply removed, and the cutis graft meshed to 5 expansion sizes (1:1, 1.5:1, 2:1, 3:1, and 4:1). These grafts will then be placed on a tensometer, and stretched to evaluate the maximum force they can withstand.

These samples will be compared to treating samples of full thickness skin grafts with ReCell to remove the epidermis. The ReCell cutis samples will be tested for tensile strength, meshed to 5 expansion sizes (1:1, 1.5:1, 2:1, 3:1, and 4:1) and Histology to evaluate the condition of cutis graft.

Without wishing to be bound by theory, at smaller expansion sizes, the graft will retain enough strength to withstand the estimated maximal force applied to the post-operative abdominal, which are in the range of 22 N/cm in cranial-caudal direction and 32 N/cm in lateral direction (4). The strength will be compared to the ReCell samples.

Once the pilot study is successful, a second animal study will be started:

The study will be conducted following an IRB approved protocol in an IACUC approved facility. The endpoints will require non-survival surgery to provide investigators necessary tissue samples. The rodent model will include ten subjects in each arm of the study with the following breakdowns:
a) Group A: Hernia model control with laparotomy and 3 plain gut fascial closure only.
b) Group B: Hernia model laparotomy with cutis graft underlay
c) Group C: Hernia model laparotomy with ReCell-treated cutis graft underlay
d) Group D: Hernia model laparotomy with Recell-treated cutis graft underlay and application of ReCell autologous skin cell suspension to fascia.
e) Group E: Hernia model laparotomy with Recell autologous skin cell suspension applied to fascia only.

Data will be collected by the co-investigators and members of the delegation log that have reviewed the protocol and have prior participation in hernia repair procedures of mammals. Analysis of the data will include descriptive statistics provided to the study sponsor.

### REFERENCES CITED IN THIS EXAMPLE

1. Swenson, S. Cutis Grafts; Application of the Dermatome-Flap Method. Arch Surg 1943 (564-570)
2. Rutter, A. G. Cyst formation seven years after a cutis graft repair of hernia, British Medical Journal Apr. 16, 1955 (951-952)
3. Peer, L., Paddock, R., Histologic studies on the fate of deeply implanted dermal grafts, JAMA June 1935 (268-290)
4. Mechanical Properties of Mesh Materials Used for Hernia Repair and Soft Tissue Augmentation Peter P. Pott, Markus L. R. Schwarz, Published: October 12, Journal of Public Library of Science

### Example 5

CutE GRAFT (Cutis ExVivo Graft Resiliency Assessment and Function Trial)

Populations: Age >18

Hernias are a protrusion of an organ through the wall of a cavity and are classified by location and/or etiology.¹ Hernia repair is the most common procedure performed with over one million surgeries in the United States and 20 million hernia repairs worldwide each year.² Hernia repairs have been a major revenue stream for U.S. hospitals at $48 billion/year and are projected to continue increasing due to obesity and increased abdominal surgeries.³⁻⁴ The repair of hernia defects involves synthetic or biologic mesh in 80% of the cases in the U.S.⁵ This mesh repair reinforces the defect to reduce tension and the risk of recurrence through fibrocollagenous incorporation of the adjacent structures.⁶ The ideal repair material would be durable, resistant to infection, and able to incorporate into the host tissue with sufficient tensile strength all while minimizing long term risk of recurrence or complication.⁷ Unfortunately, 12% of patients developing a subsequent infection and complication involving the mesh. These complications have led to complex, severe injuries, dramatically impacting patients' lives and over $1 billion in settlements in the United States.⁸

As an alternative to expensive biologic meshes or infection-prone synthetic meshes, recent surgical interest has returned to cutis grafts—an autologous, full-thickness portion of the patient's excess skin adjacent to the hernia.⁹⁻¹¹ Use of the patient's own tissue allows surgeons an alternative with a native structural tissue bearing an intact vascular system that can quickly engraft without the costs of biologic or the risks of synthetic mesh repairs. Recent rodent model studies were conducted at LSUHSC New Orleans to evaluate optimal cutis graft repair technique.¹² Additional studies have demonstrated decreased pain at three month follow-up in the cutis graft group compared to mesh with similar outcomes in seroma formation, wound infection, and recurrence at three months and at one year.¹³⁻¹⁴ As a result, surgeons at LSUHSC New Orleans have performed over 300 hernia repairs using cutis grafts ranging from small umbilical hernias to larger ventral hernias and are currently being studied for long term outcomes. Unfortunately, the technique requires removal of the epidermis with an abrasive pad resulting in trauma to the tissue and up to 30 minutes to process each graft. The epidermis must be removed as it is nonviable, keratinized cells which slows engraftment and could result in increased seroma formation. A less labor-intensive technique to delaminate the epidermis would result in better adoption of this form of hernia repair by surgeons and potentially a better graft for the patient.

The ReCell® kit by Avita Medical Limited uses an enzymatic process to remove epidermis while generating an autologous skin cell suspension of keratinocytes, fibroblasts, and melanocytes. The kit features a well that heats the enzyme to an optimal temperature with a small workstation to easily remove epidermis. The kit could be expanded to a larger size to facilitate cutis graft processing and allows the enzyme to do the labor-intensive work currently mechanically performed by the surgeon.

The first step is to histologically assess enzymatically treated to mechanically treated cutis grafts. Histologic examination allows the investigators to assess potential degradation of papillary and reticular dermis to compare overall viability of the two different methods. A control biopsy allows investigators to assess for presence of epidermis, papillary dermis, reticular dermis, and adnexal structures.

Without wishing to be bound by theory, mechanically-treated cutis grafts have similar dermal architecture to enzymatically-treated cutis grafts for the purpose of hernia repair.

Primary Objectives:

Assess the presence or absence of intact epidermis in three cutis graft specimens from the study subject donor site: control (untreated), mechanically treated (standard of care), and enzymatically treated (study).

Assess the presence or absence of intact papillary dermis in three cutis graft specimens from the study subject donor site: control (untreated), mechanically treated (standard of care), and enzymatically treated (study).

Assess the presence or absence of intact reticular dermis in three cutis graft specimens from the study subject donor site: control (untreated), mechanically treated (standard of care), and enzymatically treated (study).

Scientific benefits of study: While 1940-1950s research on cutis grafts remains an important reference, current surgical methodology and suture materials have changed. Without being bound by theory, additional information carefully examining the characteristics of cutis grafts will valuable for surgeons and will result in a technically superior method of cutis graft processing.

Data Collection and Storage:

Patients undergoing surgery consenting to study (9978-UMC-NO) to provide tissue will be included. Upon notification of surgery the week prior the study coordinator (Sham Lennard, BSN) will be present in the operating room at the time of the tissue procurement to ensure fresh tissue that is NOT frozen or treated with additional preservatives. The procuring surgeon will provide two full-thickness 1×2 cm samples to the study coordinator labeled, “Control sample,” and “Enzymatic sample,” which will later undergo processing. The third 1×2 cm sample will be labeled, “Mechanical sample” and provided by the surgeon immediately after she/he has performed the standard of care technique of mechanically prepping the cutis graft by removing the epidermis. The study coordinator will take the three specimens to the burn center lab immediately after procuring the samples and will process the “Enzymatic sample,” for 30 minutes using the provided ReCell kit per IFU including removal of epidermis. The study coordinator will then perform TWO 4 mm punch biopsies from each sample and place them in the lab-provided formalin-filled container. Each container will be labeled either “Control,” Enzymatic,” or “Mechanical” with the study site, study subject, and date. The biopsies are then mounted, sliced, and stained with hematoxylin and eosin stains on slides. The slides will then have full slide scan images taken and provided to the investigators listed in the study. Two study investigators will review digital images of the slides and classify each biopsy using the database dictionary below. At the conclusion of the study, a brief report is provided to the study sponsor.

The Following Represent Non-Exclusive, Exemplary Definitions

| | |
|---|---|
| Subject | First two digits denote study site. Last three digits denote subject enrollment number |
| Control | Untreated cutis graft with intact epidermis |
| Biopsy | |
| Mechanical | Mechanically prepared cutis graft with partial or complete loss of dermal elements |
| Biopsy | |
| Enzymatic | Enzymatically prepared cutis graft with partial or complete loss of dermal elements |
| Biopsy | |
| Epidermis | >50% Intact keratinocytes (stratum basale, spinosum, granulosum, lucidum, OR corneum) |
| | over papillary dermis without loss of papillary architecture |
| Papillary | <50% Intact keratinocytes while maintaining elements of dermal papillae architecture AND |
| | intact collagen above the rete subpapillare |
| Reticular | Absence of intact dermal papillae WITH breaks or absence of rete subpapillare without |
| | evidence of transected reticular dermis |
| Fat | Areas of transected reticular dermis or sufficient damage to reticular dermis AND/OR adnexal |
| | structures including subcuticular elements |

Data Collection:

Data collected includes the following variables from the EMR and the Trauma/Burn Registry:

1. Age

2. Gender (M/F)

3. Race (W,B,H,O)

4. Location of skin harvest

5. Name of surgeon performing the surgery

6. Name of surgeon mechanically processing the cutis graft

7. Evidence of prior trauma to the specimens such as scars, holes, or lacerations

8. Time to enzymatic treatment completion

### REFERENCES CITED IN THIS EXAMPLE

1. Williams L S, Hopper P D. Understanding Medical-Surgical Nursing. 5^{th} ed. F. A. Davis; Philadelphia, Pa., USA: 2015. P. 770.
2. May H, Spann R G. Cutis Grafts for Repair of Incisional and Recurrent Hernia. Surg Clin of North Amer 1948; 28(2):517-24.
3. Bendavid R, Abrahamson J, Arregui M E, Flament J B, Phillips E H. Abdominal Wall Hernias: Principles and Management. 1^{st} ed. Springer; New York, N.Y., USA: 2001.
4. Heniford B T. Hernia Handbook. 1^{st} ed. Carolinas HealthCare System; Charlotte, N.C., USA: 2015.
5. Kingsnorth A, Treating inguinal hernias: Open mesh Lichtenstein operation is preferred over laparoscopy. Brit Med Jour 2004; 328:59-60.
6. Li X, Kruger J A, Jor J W, Wong V, Dietz H P, Nash M P, Nielsen P M. Characterizing the ex vivo mechanical properties of synthetic mesh. Jour Mech Behav. Biomed Mater. 2014, 37:48-55.
7. Pandit A S, Henry J A. Design of surgical meshes—An engineering perspective. Technol Health Care. 2004; 12:51-65.
8. Zhu L M, Schuster P, Klinge U. Mesh Implants: An overview of crucial mesh parameters. World Jour of Gastrointestinal Surg. 2015; 10:226-236.
9. Eisele W M, Starkloff G B. The Use of Skin Grafts in Hernia Repair. Ann Surg 1951; 134(5):897-903.
10. Strahan W B. Hernial Repair by Whole-Skin Graft with Report on 413 Cases. The British Jour of Surg. 1951; 38(151):276-84.
11. Baylon K, Rodriquez-Camarillo P, Elias-Zuniga A, Diaz-Elizondo J A, Gilderson R, Lozano K. Membranes 2017; 7(3):47.
12. Becnel C, Yoo A, Short C, Islam K, Greiffenstein P, Lau F, and Hodgdon I. Incisional hernia Prevention Using Autologous Cutis Crafts in Rat Model. Jour Amer Coll Surg 2019; 229: S100.
13. Clay L, Stark B, Gunnarsson U, Strigard K. Full-thickness skin graft vs. synthetic mesh in the repair of giant incisional hernia: a randomized controlled multicenter study. Hernia 2018; 22(2):325-32.
14. Holmdahl V, Stark B, Clay L, Gunnarsson U, Strigard K. One-year outcome after repair of giant incisional hernia using synthetic mesh or full-thickness skin graft: a randomized controlled trial. Hernia 2019; 23:355-61.

### Example 6

The standard of care for the repair of abdominal wall defects has evolved to include buttressing the hernia repair with a reinforcing implant of some sort, usually a piece of mesh which is either synthetic or derived from some biological model. Generally speaking, synthetic meshes have higher risks for serious complications such as erosion into loops of bowel, and a synthetic mesh infection can add an additional 108000 USD in annual charges [Auguenstein]. Biologic implants, meanwhile, cost upwards of 25-30 USD per square cm, meaning that a piece of mesh for a large hernia could cost as much as 40,000 USD [Petro].

Currently, there are many different brands of synthetic and biologic meshes on the market in the United States, indicating that the ideal prosthesis has yet to arrive for hernia repair [Bilsel]. Such an implant should be low cost, chemically inert, resistant to mechanical strain and infection, amenable to size customization, resistant to the formation of bowel adhesions, and similar in handling characteristics to the patient's native tissues [Sankaran].

Our team of investigators has refined an innovative technique for hernia repair called “cutis autograft” which satisfies all these criteria [Mulloy]. The surgical technique consists of excising a full thickness specimen of the hernia patient's own skin, removing the epithelium from this specimen by hand through a process of mechanical abrasion, and using the resulting sheet of the patient's' own dermis as an implant to buttress the suture repair of the hernia defect. These sheets of denuded dermis have been shown to have similar efficacy as market meshes with lower complication rates and no product costs [Blatnik]. It is, quite simply, a vastly superior implant.

As originally conceived, the primary drawback of the cutis autograft operation is the need for the surgeon to mechanically remove the epithelium from the skin specimen, a process that is both frustrating and time-consuming. It requires the surgeon to stop the progress of the operation and dedicate up to 30 minutes of time toward the process of removing the epithelium on the autologous skin specimen in preparation for its use as an implant.

Without being bound by theory, point-of-care enzymatic processes capable of removing epithelium from a skin specimen can allow the surgeon to place the skin specimen in solution and return to the task of hernia dissection while the epithelium is dissolved on the surgical field's back table.

For the surgeon end-users, saving time means getting more cases done in a day, less anesthesia time for their patients, higher job satisfaction, and monetary savings. All surgeons are acutely aware that time is their only nonrenewable resource. Hospital systems stand to benefit from a faster hernia repair operation as well since it costs 90 USD per minute to run a typical operating room at University Medical Center. Taken together, the value proposition brought by incorporating an enzymatic debridement system into our team's novel hernia repair operation using cutis autograft is significant.

Market Size/Societal Need: Hernia repairs (HRs) are among the most common operations performed by surgeons worldwide. In the United States alone, approximately 300,000 and 700,000 operations are performed annually for ventral and inguinal hernias, respectively, and incisional hernia repair costs are between 6 and 10 billion USD per year [Allied].

Synthetic mesh is used in over 80% of hernia repairs with 12% of patients developing a subsequent infection and complication involving the mesh [Allied]. These complications have led to complex, severe injuries, dramatically impacting patients' lives and over $1 billion in settlements in the United States [Allied].

The fact that the lower-complication choice of biologic mesh is cost-prohibitive for hospital systems that serve underserved populations brings in the question of justice. Inequality of health care options for the uninsured or underinsured is a reality, as any surgeon who has ever been forced to choose a cheaper, less reliable technology by their hospital system's financial situation knows. A high-performing hernia repair option that is financially accessible, such as that disclosed herein, is needed.

We have recently published evidence of 97 cutis graft repairs which bolsters our confidence in this as a disruptive technology [Hodgdon]. With 98% follow up at a mean of 10 months, serious complication rates were found to be comparable to biologic meshes. The cutis graft repairs had a 3% recurrence rate (standard mesh: 0-25%), and infections were found in 9% (standard mesh: 2.7-25%) [Ozkaya, Korenko, Blatnik, Martis, Strahan, Samson, Earle, & Lindmark]. Importantly, not only did none of the infections require removal of the cutis graft, but three of the nine did not even require antibiotics to clear the infection. For this investigation, two of the five surgeons had been performing hernia repair at University Medical Center in the year prior to the introduction of the cutis graft technique with a mean mesh cost of 19,013 USD per month for 10 months. After the introduction of the cutis graft technique, this number dropped to 7,465 USD for the same time period in the following year. Annualized, this would result in a 61% reduction in their mesh costs over a full year, from 228,153 USD to 89,475 USD.

By promulgating a technology that no longer forces hernia surgeons to choose between their patient's needs and a hospital's resources, the Cutis ExVivo Graft Resiliency Assessment and Function Trial (CutEGRAFT) I has the potential to revolutionize the world of hernia repair. The cutis graft hernia repair operation is intuitive and easy to learn. Further, the enzymatic debridement system employed in the presently disclosed technology comprises a lyophilized powder with a long shelf life. The CutEGRAFT investigators continue to build their case volumes—roughly 300 total operations to date—and thus establish themselves as the world's leaders in this arena. By employing the presently disclosed system, a hernia repair surgeon must no longer choose with which set of drawbacks and disadvantages he or she wishes to contend.

Without being bound by theory, enzymatic removal of the epithelium from the dermal graft creates a dermal graft material with tensile properties that are substantially equivalent to that seen in the dermis after the standard-of-care process of mechanical deepithelialization. Briefly, in the performance of the cutis graft surgery the surgeon estimates the square area of skin needed to construct the cutis graft implant through measurements of the hernia defect. Once the amount of skin is known, the surgeon resects that quantity of skin from the patient's body and prepares it for implantation. Normal skin has a high degree of elasticity, however, and once explanted the resulting skin specimen contracts in a manner that is highly variable. Further, it is disastrous to have a cutis graft that—at the completion of preparation—is too small for a given defect. It is normal practice, therefore, for the operating surgeon to take a slightly larger skin specimen than is originally estimated to be needed and to discard any leftover skin once the hernia defect is repaired. Cutis graft hernia repair patients are made aware of this strategy at the time of their preop consenting process.

Without being bound by theory, each subject found intraoperatively to have sufficient skin remaining at the completion of sizing the cutis graft for hernia repair has 3 specimens taken from the leftover skin sample, with each specimen measuring 2×3 cm. One specimen does not undergo mechanical or enzymatic treatment and is be labeled, “untreated.” One specimen undergoes mechanical treatment to remove 90% or greater epidermis using current standard of care techniques and is labeled, “mechanically-treated.” The third specimen is treated in the research lab by the research coordinator and overseen by the two team members with expertise in use of the enzymatic epithelial debridement solution and kit (such as the AVITA ReCell® kit (H.A.P., J.E.C.). The prescribed enzyme is used to treat the skin specimen until 90% of greater epidermis separates and will be labeled, “enzymatically-treated.”Specimens are then be measured using a micrometer and undergo tensile strength testing. Data are collected using the Instron 3343 (1 kN) to assess viscoelasticity, elongation, and strain measurements. Data are acquired using Bluehill® Universal software to generate reports using tension to assess tensile strength. A total of ten subjects are utilized in this manner.

### REFERENCES CITED IN THIS EXAMPLE

Augenstein V A, Colavita P D, Wormer B A, et al. CeDAR: Carolinas equation for determining associated risks. J Am Coll Surg. 2015; 221(4):565-566.
Petro C C, Rosen M J. A current review of long-acting resorbable meshes in abdominal wall reconstruction. Plast Reconstr Surg. 2018; 142(3 Suppl):845-91.
Bilsel Y, Abci I. The search for ideal hernia repair; mesh materials and types. Int J Surg. 2012; 10(6):317-321.
Shankaran V, Weber D J, Reed R L, Luchette F A. A review of available prosthetics for ventral hernia repair. Ann Surg. 2011; 253(1):16-26.
Mulloy C, Paige J, Hodgdon I, Cook M W. Use of Cutis Graft as Biologic Prosthesis in Complicated Abdominal Closures: A Case Report. J Clin Med Case Rep. 2020; 1(1): 2-3.
Blatnik J, Jin J, Rosen M. Abdominal hernia repair with bridging acellular dermal matrix—an expensive hernia sac. Am J Surg. 2008; 196(1):47-50.
Allied Analytics LLP. Hernia Repair Devices and Consumables Market by Product, Surgery Type, and Hernia Type—Global Opportunity Analysis and Industry Forecast, 2016-2023. Accessed 16 Feb. 2017. https://www.alliedmarketresearch.com/hernia-repair-devices-market
Hodgdon I, Cook M, Yoo A, Rajo M, Dooley D, Haydel A, Dogar S, Greiffenstein P, Morrison J, Lau F, Paige J. The Use of Autologous Fenestrated Cutis Grafts in Hernia Repair: Surgical Outcomes and Cost Analysis of 97 Consecutive Patients. In press.
Özkaya Mutlu Özay, Egemen O, Akan A, et al. The use of dermal automesh for incidental hernia repair in abdominoplasty: clinical, biochemical, and radiological results. J Plast Surg Hand Surg. 2015; 49(3):172-176.
Korenkov M, Sauerland S, Arndt M, Bograd L, Neugebauer E A M, Troidl H. Randomized clinical trial of suture repair, polypropylene mesh or autodermal hernioplasty for incisional hernia. Br J Surg. 2002; 89(1):50-56.
Blatnik J, Jin J, Rosen M. Abdominal hernia repair with bridging acellular dermal matrix—an expensive hernia sac. Am J Surg. 2008; 196(1):47-50.
Martis G, Damjanovich L. Use of double-layer autologous dermal flap in the treatment of recurrent and/or infected incisional hernias: presentation of the surgical technique and the results of 1-year follow-up—a prospective, consecutive cohort study. Hernia. 2016; 20(3):461-470.
Strahan A W B. Hernial repair by whole-skin graft, with report on 413 cases. Br J Surg. 1951; 38(151):276-284.
Samson T D, Buchel E W, Garvey P B. Repair of infected abdominal wall hernias in obese patients using autologous dermal grafts for reinforcement. Plast Reconstr Surg. 2005; 116(2):523-527.
Earle A S, Abdel-Fattah M A. Closure of an abdominal hernia with a groin flap lined with a dermal graft. Case report. Plast Reconstr Surg. 1975; 56(4):447-449.
Lindmark M, Strigard K, Löwenmark T, Dahlstrand U, Gunnarsson U. Risk factors for surgical complications in ventral hernia repair. World J Surg. 2018; 42(11):3528-3536.
Buckley J, Elliott R S, Seifi A. Usage of Skin Graft Procedures during the Past Two Decades in the United States. Academic Surgical Congress. 2019; Abstract 84.11.

### Example 7

Without being bound by theory, the enzymatic processing of full thickness skin grafts into cutis grafts is effective, does not degrade the quality of the repair, and alleviates the extra burden on the surgeon from mechanical debridement.

Without wising to be bound by theory, processing the skin with enzyme results in (1) removal of the epidermis without significant degradation of the dermis, (2) (1) more uniform removal of the dermis, and (2) enhanced preservation of the papillary dermis as compared to mechanical debridement.

Without being bound by theory, processing the skin with enzyme does not degrade the tensile strength of skin to the point it is rendered worthless for repair, and enzymatic treatment is stronger than mechanically treated skin. Holmdahl et al. presents one exemplary method and model for assessing tensile strength of skin grafts for use in hernia repair.¹ ¹ Holmdahl V, Backman O, Gunnarsson U, Strigard K. The Tensile Strength of Full-Thickness Skin: A Laboratory Study Prior to Its Use as Reinforcement in Parastomal Hernia Repair. Front Surg. 2019; 6:69.

Further, without wishing to be bound by theory:

after enzymatic degradation of the graft, dilution effectively removes the enzyme from the resultant dermal graft such that implantation of the enzymatically processed grafts does not degrade the repair site;

hernia repair with enzymatically processed dermal grafts produces reduced or similar recurrence rates in subjects as compared to mechanically processed cutis grafts and existing mechanisms of hernia repair (such as via use of synthetic or biological meshes); and

enzymatic treatment and use of cutis grafts are able to eliminate and/or greatly reduce the 2% cyst formation rate as reported from a 1950s era inguinal hernia study using full thickness skin grafts.

WI though being bound by theory, enzymatic processing results in uniform removal of the epidermis with maximal preservation of papillary dermis resulting in a cutis graft that is equivalent in tensile strength to Full Thickness Skin Graft (FTSG), and the level of uniformity is superior to mechanically processed cutis as the mechanical process does not effectively remove the epidermis in all areas and results in tissue weakening in other areas. Further, and without wishing to be bound by theory, after processing, the enzyme is diluted and or denatured well enough to prevent any detrimental effects of the residual enzyme to the patient or the hernia repair. Finally, without being bound by theory, repairs using enzymatically processed cutis grafts are superior to FTSG in that the cyst formation rate is reduced to negligible levels, with equivalent results in terms of recurrence and performance in infected wounds.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A method of preventing the development of a hernia within a subject at risk of developing a hernia, comprising:
selecting a graft material, wherein the graft material comprises an epidermis and a dermis;
removing the epidermis from the graft material through enzymatic processing, thereby providing a dermal graft material substantially comprising dermis; and
implanting the dermal graft material in contact with an opening in the abdominal wall, wherein the dermal graft material repairs the abdominal wall opening, thereby preventing the development of a hernia in the subject.

2. A method of repairing a hernia, the method comprising:
selecting a graft material, wherein the graft material comprises an epidermis and a dermis;
removing the epidermis from the graft material through enzymatic processing, thereby providing a dermal graft material; and
implanting the dermal graft material in contact with an opening in the abdominal wall, wherein the dermal graft material repairs the abdominal wall opening.

3. The method of claim 1 or claim 2, wherein the graft material comprises a full-thickness autologous skin graft.

4. The method of claim 1 or claim 2, further comprising the step of perforating the graft material to permit efflux of serous fluid.

5. The method of claim 1 or claim 2, further comprising the steps of
collecting an epidermal cellular solution following enzymatic processing, wherein the epidermal solution substantially comprises the epidermis that was subjected to enzymatic processing; and
applying the epidermal cellular solution to the abdominal wall opening, wherein the epidermal cellular solution promotes healing of the abdominal wall opening.

6. The method of claim 1 or claim 2, wherein the enzymatic processing comprises exposure of the graft material to an enzymatic solution that is devoid of xenogeneic components, allogeneic components, or a combination thereof.

7. The method of claim 1 or claim 2, wherein an enzyme used in the enzymatic processing is configured to disrupt disulfide bonds.

8. The method of claim 1 or claim 2, wherein the enzyme comprises trypsin, dispase, collagenase, trypsin-EDTA, thermolysin, pronase, hyaluronidase, elastase, papain, pancreatin, DNAase, or a combination thereof.

9. The method of claim 7, wherein the enzyme comprises a RECELL® Enzyme.

10. The method of claim 1 or claim 2, wherein the dermal graft material comprises a cellular suspension of dermal cells capable of reproduction.

11. The method of claim 10, wherein the step of implanting the dermal graft material comprises spraying the cellular suspension onto, in close proximity to, or adjacent to the abdominal wall opening.

12. The method of claim 1 or claim 2, wherein the dermal graft material is aligned with the opening in the abdominal wall.

13. The method of claim 1 or claim 2, wherein the opening comprises a surgical incision.

14. The method of claim 1 or claim 2, wherein the opening comprises debrided fascia.

15. The method of claim 1 or claim 2, wherein the dermal graft material promotes healing of abdominal fascial edges.

16. The method of claim 1 or claim 2, wherein the dermal graft material is not actively affixed to the abdominal wall.

17. The method of claim 1 or claim 2, wherein the dermal graft material is anchored to the abdominal wall by pressure, an adhesive, a clip, a tack, a suture, a staple, a screw, or a combination thereof.

18. The method of claim 1 or claim 2, wherein the dermal graft material is implanted over or ventral to the abdominal wall opening.

19. The method of claim 1 or claim 2, wherein the dermal graft material is implanted under or dorsal to the abdominal wall opening.

20. The method of claim 1 or claim 2, further comprising substantially or completely closing the abdominal wall opening prior to implanting the dermal graft material.

21. The method of claim 1 or claim 2, further comprising substantially or completely closing the abdominal wall opening after implanting the dermal graft material.

22. The method of claim 20 or 21, wherein the abdominal wall opening is closed with synthetic mesh or biological mesh.

23. The method of claim 1 or claim 2, wherein the abdominal wall opening comprises a surgical incision.

24. The method of claim 23, wherein the surgical incision comprises an abdominal fascia incision.

25. The method of claim 1 or claim 2, wherein the abdominal wall opening is caused by surgery.

26. The method of claim 25, wherein the surgery comprises laparotomy (celiotomy), stoma surgery, or repair of abdominal hernia.

27. The method of claim 26, wherein the hernia comprises an incisional hernia, ventral hernia, umbilical hernia, epigastric hernia, lumbar hernia, inguinal hernia, diaphragmatic hernia, hiatal hernia, Spigelian hernia, or a parastomal hernia.

28. The method of claim 1 or claim 2, wherein the subject at risk of developing a hernia comprises an obese subject, a subject afflicted with an infection, a subject who underwent a bowel resection, a subject who underwent colon surgery, a subject being administered corticosteroids, a subject who smokes, a subject who has chronic obstructive pulmonary disease or any combination thereof.

29. The method of claim 28, wherein the infection comprises a surgical site infection, intra-abdominal infection, deep infections, or superficial abdominal infection.

30. The method of claim 1 or claim 2, wherein the graft material comprises a mammalian tissue.

31. The method of claim 30, wherein the mammalian tissue comprises endogenous growth factors.

32. The method of claim 1 or claim 2, wherein the graft material comprises a dehydrated tissue, decellularized tissue, cross-linked tissue, frozen tissue, cryopreserved tissue, fresh tissue, or any combination thereof.

33. The method of claim 1 or claim 2, wherein the graft material is implanted as a sheet, nanoparticle, powder, or injectable.

34. The method of claim 1 or claim 2, wherein the graft material further comprises a synthetic mesh, biological mesh, or tissue scaffold.

35. The method of claim 34, wherein the biological mesh comprises a mammalian tissue.

36. The method of claim 35, wherein the mammalian tissue comprises a dermal matrix, or a urinary bladder matrix.

37. The method of claim 35, wherein the mammalian tissue scaffold comprises a collagen matrix.

38. A method of producing a dermal graft material comprising:
obtaining a graft material that comprises an epidermis and a dermis;
disaggregating epidermis from the graft material, wherein the step of disaggregating the epidermis from the graft material comprises subjecting the graft material to enzymatic processing, thereby providing a dermal graft material.

39. The method of claim 38, wherein the graft material comprises full-thickness skin graft.

40. A method of preventing cyst formation following implantation of a dermal graft material within a subject, comprising:
selecting a graft material, wherein the graft material comprises an epidermis and a dermis;
removing the epidermis from the graft material through enzymatic processing, thereby providing the dermal graft material; and
implanting the dermal graft material,
wherein the enzymatic processing prevents cyst formation following implantation of the dermal graft material.

41. The method of claim 38 or claim 40, wherein the dermal graft material comprises a cutis graft.

42. The method of claim 38 or claim 40, wherein the dermal graft material comprises an autologous dermal graft.

43. The method of claim 38 or claim 40, wherein the graft material comprises a full-thickness skin graft.

44. A method of treating an internal wound, comprising:
selecting a graft material, wherein the graft material comprises an epidermis and a dermis;
removing the epidermis from the graft material through enzymatic processing, thereby providing a dermal graft material; and
implanting the dermal graft material over an internal wound of a subject.

45. The method of claim 44, wherein the graft material comprises a cutis graft.

46. The method of claim 44, wherein the graft material comprises an autologous dermal graft material.

47. The method of claim 44, wherein the internal wound comprises an injury to a tendon, a fistula, gastrotomy, enterotomy, colotomy, bile duct injury, bladder injury, ureteral injury, arterial or venous injuries, serosal tears, or a combination thereof.

48. The method of claim 47, wherein the fistula comprises an anal fistula, colorectal fistula, enterocutaneous fistula, choledochocutaneous fistula, cholecystocutaneous fistula, gastrocutaneous fistula, esophagocutaneous fistula, gastroatmospheric fistula, enteroatmospheric fistula, coloatmospheric fistula, rectoatmospheric fistula, gastrogastric fistula, gastroenteric fistula, gastrocolic fistula, enteroenteric fistula, enterocolic fistula, colovaginal fistula, colovesical fistula, rectovaginal fistula, rectovesical fistula, colourethral fistula, rectourethral fistula, tracheoesophageal fistula, bronchoesophageal fistula, tracheopleural fistula, bronchopleural fistula, aortoenteric fistula, or a combination thereof.

49. A method of promoting healing of an opening in the abdominal wall, comprising:
obtaining a graft material, wherein the graft material comprises an epidermis and a dermis;
removing the epidermis from the graft material through enzymatic processing, thereby providing a dermal graft material; and
implanting the dermal graft material in a subject, wherein the dermal graft material is implanted in contact with the opening in the abdominal wall, and wherein the dermal graft material promotes healing of the abdominal wall opening.

50. A method of promoting fascial healing, comprising:
obtaining a graft material, wherein the graft material comprises an epidermis and a dermis;
removing the epidermis from the graft material through enzymatic processing, thereby providing a dermal graft material; and
implanting dermal graft material in a subject, wherein the dermal graft material is implanted approximately to a defective region in the fascia, and wherein the dermal graft material promotes healing of the defect in the fascia.

51. A method of preventing hernia recurrence, comprising:
obtaining a graft material, wherein the graft material comprises an epidermis and a dermis;
removing the epidermis from the graft material through enzymatic processing, thereby providing a dermal graft material; and
implanting the dermal graft material in a subject, wherein the dermal graft material is implanted in contact with an opening in the abdominal wall, and wherein the dermal graft material prevents hernia recurrence.

52. A kit for producing a dermal graft, the kit comprising:
an enzymatic solution configured to disaggregate the epidermis from a full-thickness skin graft, thereby producing a dermal graft material; and
instructions for use.

53. A method of treating a medical condition with a regenerative cell suspension, the method comprising:
obtaining an epidermal solution, wherein the epidermal solution comprises autologous epidermal cells from a subject suffering from the medical condition; and
applying the epidermal solution to an area afflicted with the medical condition,
wherein the epidermal solution treats the medical condition.

54. The method of claim 53, further comprising implanting a dermal graft material to the area afflicted with the medial condition, wherein the dermal graft material and the epidermal solution treat the medical condition.

55. The method of any one of claims 1, 2, 38, 40, 44, 49, 50, 51, and 53, wherein the dermal graft comprises a tensile strength of between about 50 N/cm to about 800 N/cm.

56. The method of any one of claims 1, 2, 38, 40, 44, 49, 50, 51, and 53, wherein the dermal graft comprises a tensile strength of up to about 700 N/cm.

57. The method of any one of claims 1, 2, 38, 40, 44, 49, 50, 51, and 53, wherein the dermal graft comprises a tensile strength of about 600 N/cm.

58. A method of creating a regenerative cell suspension for the treatment of a medical condition, the method comprising:
applying an enzymatic solution to an epidermis of a subject suffering from the medical condition, wherein the enzymatic solution is configured to disaggregate the epidermis from a dermis of the subject;
permitting the enzymatic solution to disaggregate the epidermis from the dermis to form an epidermal solution; and
collecting the epidermal solution, wherein the collected epidermal solution forms the regenerative cell suspension.

59. The method of claim 51 or claim 58, wherein the enzymatic solution is applied to epidermis from an autologous, full-thickness skin graft obtained from the subject.

60. The method of claim 53 or 58, wherein the medical condition comprises chronic wounds, exfoliative skin diseases, a surgical wound, or a combination thereof.

61. A regenerative cell suspension configured to treat a medical condition comprising:
an epidermal solution, wherein the epidermal solution comprises autologous epidermal cells from a subject afflicted with the medical condition.

62. The regenerative cell suspension of claim 61, wherein the medical condition comprises chronic wounds, exfoliative skin diseases, a surgical wound, or a combination thereof.

63. A method of preventing the development of a hernia within a subject at risk of developing a hernia, comprising:
selecting a graft material, wherein the graft material comprises an epidermis and a dermis;
removing the epidermis from the graft material through enzymatic processing,
collecting an epidermal solution following enzymatic processing, wherein the epidermal solution comprises the epidermis that was subjected to enzymatic processing; and
applying the epidermal solution to an abdominal wall opening, wherein the epidermal solution promotes healing of the abdominal wall opening.

64. An enzymatic debridement system for use in creating a dermal graft from a graft material when the graft material comprises an epidermis and a dermis, the system comprising:
an enzymatic solution configured to remove substantially all of the epidermis from the graft material when exposed thereto.

65. The enzymatic debridement system of claim 64, wherein the enzymatic solution is devoid of xenogenic components, allogentic components, or a combination thereof.

66. The enzymatic debridement system of claim 64, wherein an enzyme used in the enzymatic processing is configured to disrupt disulfide bonds.

67. The enzymatic debridement system of claim 64, wherein the enzyme comprises trypsin, dispase, collagenase, trypsin-EDTA, thermolysin, pronase, hyaluronidase, elastase, papain, pancreatin, DNAase, or a combination thereof.

68. The enzymatic debridement system of claim 67, wherein the enzyme comprises a RECELL® Enzyme.

69. The enzymatic debridement system of claim 64, wherein the graft material comprises a mammalian tissue.

70. The enzymatic debridement system of claim 69, wherein the mammalian tissue comprises endogenous growth factors.

71. The enzymatic debridement system of claim 64, wherein the graft material comprises a dehydrated tissue, decellularized tissue, cross-linked tissue, frozen tissue, cryopreserved tissue, fresh tissue, or any combination thereof.

72. The enzymatic debridement system of claim 64, wherein the dermal graft material comprises a cutis graft.

73. The enzymatic debridement system of claim 64, wherein the graft material comprises an autologous graft.

74. The enzymatic debridement system of claim 64, wherein the graft material comprises a full-thickness skin graft.

75. The enzymatic debridement system of claim 64, wherein the dermal graft comprises a tensile strength that is substantially equivalent the tensile strength of a full thickness skin graft.

76. The enzymatic debridement system of claim 64, wherein the dermal graft comprises a tensile strength of between about 50 N/cm to about 800 N/cm.

77. The enzymatic debridement system of claim 64, wherein the dermal graft comprises a tensile strength of up to about 700 N/cm.

78. The enzymatic debridement system of claim 64, wherein the dermal graft comprises a tensile strength of about 600 N/cm.

79. The enzymatic debridement system of claim 64, further comprising a lyophilized powder configured to extend a shelf life of the resultant dermal graft.
